(19) United States
(12) Patent Application Publication
(10) Pub. No.: US 2025/0014723 A1
(43) Pub. Date: Jan. 9, 2025

(54) SURGICAL SYSTEMS WITH INTERACTIVE HANDLING AND DISPOSAL GUIDANCE

(71) Applicant: Cilag GmbH International, Zug (CH)

(72) Inventors: Shane R. Adams, Lebanon, OH (US); Frederick E. Shelton, IV, Hillsboro, OH (US); Jacqueline C. Aronhalt, Loveland, OH (US); James Hoffmaster, Sharonville, OH (US); Kevin M. Fiebig, Cincinnati, OH (US); Matthew D. Cowperthwait, Cincinnati, OH (US); Taylor W. Aronhalt, Loveland, OH (US)

(21) Appl. No.: 18/452,928

(22) Filed: Aug. 21, 2023

Related U.S. Application Data
(60) Provisional application No. 63/525,572, filed on Jul. 7, 2023.

(51) Int. Cl. G16H 40/20; G08B 5/36

(52) U.S. Cl. CPC G16H 40/20; G08B 5/36

(57) ABSTRACT

Surgical systems and methods are provided. The surgical systems include a manufacturer-sealed sterile surgical package containing a surgical instrument configured to be used in a surgical procedure, at least one data processor, and memory storing instructions configured to cause the at least one data processor to perform operations. The operations include catalogue components of the surgical instrument, determine disposal procedures for each of the components of the surgical instrument, and provide the disposal procedures at the end of the surgical procedure.

### CROSS-REFERENCE TO RELATED APPLICATION

This application claims the benefit of and priority to U.S. Provisional Application No. 63/525,572, filed Jul. 7, 2023, the contents of which is herein incorporated by reference in its entirety.

### FIELD

The present disclosure relates generally to smart surgical devices, systems, and methods.

### BACKGROUND

Surgical instruments are manufactured to meet specific performance metrics in order to ultimately perform a surgical procedure in a safe and effective manner. Following manufacture, surgical instruments are shipped all over the world to hospitals and surgical centers, and along the supply chain, the surgical instruments are handled by a variety of persons, including transit operators, warehouse operators, customs officials, hospital staff, surgical teams, and more.

While safe and effective performance of the surgical instrument is the goal, problems can arise. In some cases, problems with the surgical instrument may arise due to manufacturing issues. In other cases, problems may arise as a result of mishandling during transit, such as improper storage techniques or extreme environmental phenomena. In still other cases, problems may arise as a result of incompatibility with other surgical equipment or improper usage in an operating room during a surgical procedure.

Accordingly, there remains a need for improved surgical systems and methods.

### SUMMARY

Surgical systems and methods are provided. In one embodiment, a surgical system is provided that includes a manufacturer-sealed sterile surgical package containing a surgical instrument configured to be used in a surgical procedure, at least one data processor, and memory storing instructions. The instructions can be configured to cause the at least one data processor to perform operations including to catalogue components of the surgical instrument, determine disposal procedures for each of the components of the surgical instrument, and provide the disposal procedures at the end of the surgical procedure.

The surgical system can vary in a number of ways. For example, the memory can store instructions configured to cause the at least one data processor to perform operations including track the disposal procedures for each component of the surgical instrument, determine an accuracy level of the disposal procedures for each tracked component, provide the accuracy level. In other aspects, the memory can store instructions configured to cause the at least one data processor to perform operations including receive data characterizing available disposal options at a healthcare site hosting the surgical procedure, adjust the disposal procedures based on the received data, and provide the adjusted disposal procedures. In other embodiments, the memory can store instructions configured to cause the at least one data processor to perform operations including receive data characterizing a geopolitical location of a healthcare site hosting the surgical procedure, adjust the disposal procedures based on the received data, and provide the adjusted disposal procedures. In other aspects, the memory can store instructions configured to cause the at least one data processor to perform operations including receive data characterizing a performance error of the surgical instrument, adjust the disposal procedures based on the received data, and provide the adjusted disposal procedures. In some variations, the adjusted disposal procedures can include a return address for the surgical instrument. The guidance can include instructions for disassembly, compartmentalization, and/or separation of the catalogued components of the surgical procedure. In other embodiments, the disposal procedures can be provided via a display located on the manufacturer-sealed sterile surgical package, such as an e-ink label and/or a colored LED located on the manufacturer-sealed sterile surgical package, with a color of the colored LED corresponding to a color of one or more disposal bins located within an operating room hosting the surgical procedure. In other embodiments, the disposal procedures can be transmitted and displayed on a remote device in electronic communication with the manufacturer-sealed sterile surgical package.

In another embodiment, a method is provided including cataloguing, by a manufacturer-sealed sterile surgical package, components of a surgical instrument contained within the manufacturer-sealed sterile surgical package and used in a surgical procedure, determining disposal procedures for each of the components of the surgical instrument, and providing the disposal procedures at the end of the surgical procedure.

The method can vary in a number of ways. For example, the method can include tracking the disposal procedures for each component of the surgical instrument, determining an accuracy level of the disposal procedures for each tracked component, and providing the accuracy level. In some variations, the method can include transmitting data characterizing at least one warning message when the accuracy level falls below a predetermined threshold. For example, the method can include receiving data characterizing available disposal options at a healthcare site hosting the surgical procedure, adjusting the disposal procedures based on the received data, and providing the adjusted disposal procedures. In other embodiments, the method can include receiving data characterizing a geopolitical location of a healthcare site hosting the surgical procedure, adjusting the disposal procedures based on the received data, and providing the adjusted disposal procedures. In other aspects, the method can include receiving data characterizing a performance error of the surgical instrument, adjusting the disposal procedures based on the received data, and providing the adjusted disposal procedures. In some variations, the adjusted disposal procedures can include a return address for the surgical instrument. The guidance can include instructions for disassembly, compartmentalization, and/or separation of the catalogued components of the surgical procedure. In other embodiments, the disposal procedures can be provided via a display located on the manufacturer-sealed sterile surgical package, such as a colored LED located on the manufacturer-sealed sterile surgical package. A color of the colored LED can correspond to a color of one or more disposal bins.

Non-transitory computer program products are provided. In one embodiment, a non-transitory computer program product is provided and can store instructions which, when executed by at least one data processor forming part of at least one computing system, cause the at least one data processor to implement operations including cataloguing, by a manufacturer-sealed sterile surgical package, components of a surgical instrument contained within the manufacturer-sealed sterile surgical package and used in a surgical procedure, determining disposal procedures for each of the components of the surgical instrument, and providing the disposal procedures at the end of the surgical procedure.

Surgical systems are also provided. In one embodiment, a surgical system is provided that includes a manufacturer-sealed sterile surgical package containing a surgical instrument, at least one data processor, and memory storing instructions configured to cause the at least one data processor to perform operations. The operations can include updating a historical record of the surgical package and/or the surgical instrument based on at least one of a current geo-political location and a supply-chain progression of the surgical system, and providing, based on at least one of the current geo-political location and the supply-chain progression of the surgical system, handling instructions for the surgical system.

The surgical system can vary in a number of ways. For example, the handling instructions can include information characterizing at least one of historic states, future states, and operational information. In some variations, the historic states can include at least one of manufacturing information, materials and components information, geographical origin information, and environmental information experienced by the surgical package. In other variations, the future states can include at least one of an intended destination information, environmental requirements information, customs information, disposal information, and error reporting procedures information. In further variations, the operational information can include at least one of product compatibility information, expected lifecycle information, and operational instructions. For example, the operations can include providing, based on an on-demand user request, at least a portion of the historical record. In some variations, the on-demand user request can be pre-authenticated. For example, the handling instructions can be provided via a display located on the manufacturer-sealed sterile surgical package. For example, the handling instructions can include a list of one or more surgical items for a surgical procedure involving the surgical instrument.

In another embodiment, a method is provided and can include updating, with a surgical system, a historical record of the surgical system based on at least one of a current geo-political location and a supply-chain progression of the surgical system, and providing, with the surgical system and based on at least one of the current geo-political location and the supply-chain progression of the surgical system, handling instructions for the surgical system. The surgical system can include a manufacturer-sealed sterile surgical package containing a surgical instrument.

The method can vary in a number of ways. For example, the handling instructions can include information characterizing at least one of historic states, future states, and operational information. In some variations, the historic states can include at least one of manufacturing information, materials and components information, geographical origin information, and environmental information experienced by the surgical package. The future states can include at least one of an intended destination information, environmental requirements information, customs information, disposal information, and error reporting procedures information. The operational information can include at least one of product compatibility information, expected lifecycle information, and operational instructions. The method can further include providing, based on an on-demand user request, at least a portion of the historical record. In some variations, the on-demand user request is authenticated prior to providing the at least a portion of the historical record. In other aspects, the handling instructions can be provided via a display located on the manufacturer-sealed sterile surgical package. For example, the handling instructions can include a list of one or more surgical items for a surgical procedure involving the surgical instrument. In some variations, the method can further include transmitting at least one message indicating the absence of at least one of the one or more surgical items.

In another embodiment, a non-transitory computer program product is provided. The non-transitory computer program product can store instructions that, when executed by at least one data processor forming part of at least one computing system, cause the at least one data processor to implement operations. The operations can include updating, with a surgical system, a historical record of the surgical system based on at least one of a current geo-political location and a supply-chain progression of the surgical system, and providing, with the surgical system and based on at least one of the current geo-political location and the supply-chain progression of the surgical system, handling instructions for the surgical system. The surgical system can include a manufacturer-sealed sterile surgical package containing a surgical instrument.

In another embodiment, a surgical system is provided. The surgical system can include at least one manufacturer-sealed sterile surgical package containing a surgical instrument, and an electronic management system in electronic communication with the at least one manufacturer-sealed sterile surgical package. The electronic management system can be configured to receive inventory data characterizing inventory information of the manufacturer-sealed sterile surgical package, update a master management list with the received inventory data, determine, based on the updated master management list, a procedure inventory list for a surgical procedure utilizing the surgical instrument, and provide the procedure inventory list. The procedure inventory list can include surgical equipment needed for the surgical procedure.

The surgical system can vary in a number of ways. For example, the surgical system can include receiving, upon initial detection of the at least one surgical system by the network system, initial inventory data characterizing inventory information of the detected surgical system, and appending the master management list with the initial inventory data. For example, the inventory information can include a current location of the at least one surgical system. In some variations, the inventory information can be received at periodic intervals. For example, providing, upon detection that a first portion of the surgical equipment on the procedure inventory list is in a first location, instructions to transport the first portion to a second location. In some variations, the surgical system can include providing, upon detection that a second portion of the surgical equipment on the procedure list is in a third location, instructions to transport the second portion to the second location. In some variations, the first location can be a first stockroom within a hospital and the second location is an operating room within the hospital. In other variations, the first location can be a first stockroom within a hospital, the second location can be an operating room within the hospital, and the third location can be a second stockroom within the hospital.

In another embodiment, a method is provided and can include receiving inventory data characterizing inventory information of at least one surgical system in electronic communication with an electronic management system, updating a master management list with the received inventory data, determining, based on the updated master management list, a procedure inventory list for a surgical procedure utilizing the surgical instrument, and providing the procedure inventory list. The surgical system can include a manufacturer-sealed sterile surgical package containing a surgical instrument. The procedure inventory list can include surgical equipment needed for the surgical procedure.

The method can vary in a number of ways. For example, the method can include receiving, upon initial detection of the at least one surgical system by the network system, initial inventory data characterizing inventory information of the detected surgical system, and appending the master management list with the initial inventory data. The inventory information can include a current location of the at least one surgical system. In some variations, the inventory information can be received at periodic intervals. The method can also include providing, upon detection that a first portion of the surgical equipment on the procedure inventory list is in a first location, instructions to transport the first portion to a second location. In some variations, the method can include providing, upon detection that a second portion of the surgical equipment on the procedure list is in a third location, instructions to transport the second portion to the second location. The first location can be a first stock room within a hospital and the second location can be an operating room within the hospital. The third location can be a second stockroom within the hospital.

In one embodiment, a non-transitory computer program product is provided. The non-transitory computer program product can store instructions which, when executed by at least one data processor forming part of at least one computing system, cause the at least one data processor to implement operations. The operations can include receiving inventory data characterizing inventory information of at least one surgical system in electronic communication with an electronic management system, updating a master management list with the received inventory data, determining, based on the updated master management list, a procedure inventory list for a surgical procedure utilizing the surgical instrument, and providing the procedure inventory list. The surgical system can include a manufacturer-sealed sterile surgical package containing a surgical instrument. The procedure inventory list including surgical equipment needed for the surgical procedure.

In another embodiment, a surgical system is provided and can include at least one primary surgical system, and an electronic management system in electronic communication with the primary surgical system. The electronic management system can be configured to update a virtual compatibility list stored on a primary surgical system with compatibility information. The compatibility information can characterize a level of compatibility between the primary surgical system and one or more secondary surgical systems in electronic communication with the electronic management system, create a first virtual kit for a surgical procedure, and provide the first virtual kit. The first virtual kit can include the primary surgical system and at least one of the one or more secondary surgical systems selected based upon the compatibility list.

The surgical system can vary in a number of ways. For example, the primary surgical system can include a manufacturer-sealed sterile surgical package containing a surgical instrument. The electronic management server can be configured to append the first virtual kit with one or more tertiary systems. The one or more tertiary systems can be determined based on a preference of a surgeon performing the surgical procedure. In other aspects, the electronic management server, after receiving a notice characterizing an error concerning the selected at least one of the one or more secondary surgical systems, can be configured to swap the selected at least one of the one or more secondary surgical systems with another of the one or more secondary surgical systems based on the compatibility list. In some variations, the error can be a lack of available inventory of the selected at least one of the one or more secondary surgical systems. For example, the electronic management server can be configured to create a second virtual kit for a second surgical procedure. The second virtual kit can include the primary surgical system and at least one of the one or more secondary surgical systems selected based upon the compatibility list. In some variations, the first surgical procedure and the second surgical procedure can be different types of surgical procedures. In other embodiments, the electronic management server can be further configured to order the surgical systems listed in the first virtual kit from a supplier.

In another embodiment, a method is provided that includes updating a virtual compatibility list stored on a primary surgical system in electronic communication with a management server with compatibility information, creating a first virtual kit for a surgical procedure, and providing the first virtual kit. The compatibility information can characterize a level of compatibility between the primary surgical system and one or more secondary surgical systems in electronic communication with the management server. The first virtual kit can include the primary surgical system and at least one of the one or more secondary surgical systems selected based upon the compatibility list.

The method can vary in a number of ways. For example, the primary surgical system can include a manufacturer-sealed sterile surgical package containing a surgical instrument. The method can further include appending the first virtual kit with one or more tertiary systems that are determined based on a preference of a surgeon performing the surgical procedure. The method can further include swapping, after receiving a notice characterizing an error concerning the selected at least one of the one or more secondary surgical systems, the selected at least one of the one or more secondary surgical systems with another of the one or more secondary surgical systems based on the compatibility list. In some variations, the error can be a lack of available inventory of the selected at least one of the one or more secondary surgical systems. The method can further include creating a second virtual kit for a second surgical procedure. The second virtual kit can include the primary surgical system and at least one of the one or more secondary surgical systems selected based upon the compatibility list. In some variations, the first surgical procedure and the second surgical procedure can be different types of surgical procedures. For example, the method can include ordering the surgical systems listed in the first virtual kit from a supplier.

In another embodiment, a non-transitory computer program product is provided. The non-transitory computer program product can store instructions that, when executed by at least one data processor forming part of at least one computing system, cause the at least one data processor to implement operations. The operations can include updating a virtual compatibility list stored on a primary surgical system in electronic communication with a management server with compatibility information, creating a first virtual kit for a surgical procedure, and providing the first virtual kit. The compatibility information can characterize a level of compatibility between the primary surgical system and one or more secondary surgical systems in electronic communication with the management server. The first virtual kit can include the primary surgical system and at least one of the one or more secondary surgical systems selected based upon the compatibility list.

In another embodiment, a surgical system is provided and can include a manufacturer-sealed, sterile surgical package containing a surgical instrument, at least one data processor, and a memory storing instructions configured to, prior to breach of the manufacturer-sealed sterile surgical package, cause the at least one data processor to perform operations. The operations can include receive, from a remote server, data characterizing a geo-political location of at least one of the surgical package and the surgical instrument, and transmit instructions, based on the received data, to at least one of the surgical package and the surgical instrument to alter one or more aspects of the surgical package and/or the surgical instrument.

The surgical system can vary in a number of ways. For example, the manufacturer-sealed sterile surgical package can include a display configured to present visual information. In some variations, the display can be configured to present one or more messages indicating non-compliance with one or more regulations of the geo-political location. In some aspects, a language of the one or more presented messages can be based upon the geo-political location. For example, the one or more aspects can include the capacity to collect medical data and data related to surgical procedures. The instructions can be configured to disable one or more functions of the surgical package. The one or more functions can be non-compliant with one or more regulations of the geo-political location. The one or more aspects can include one or more operations of the surgical instrument usable during a surgical procedure. For example, the geo-political location can be a customs checkpoint. In other embodiments, the one or more aspects can include the capacity to deliver one or more controlled substances, and the one or more controlled substances can be prohibited or controlled in the geo-political location.

In another embodiment, a method is provided. The method can include receiving, by a surgical system and from a remote server, data characterizing a geo-political location of the surgical system, and transmitting instructions, based on the received data, to the surgical package to selectively alter one or more aspects of the surgical package. The surgical system can include a manufacturer-sealed, sterile surgical package and a surgical instrument contained within the manufacturer-sealed, sterile surgical package.

The method can vary in a number of ways. For example, the manufacturer-sealed sterile surgical package can include a display configured to present visual information. In some variations, the display can be configured to present one or more messages indicating non-compliance with one or more regulations of the geo-political location. In some variations, a language of the one or more presented messages can be based upon the geo-political location. The one or more aspects can include the capacity to collect medical data and data related to surgical procedures. The instructions can disable one or more functions of the surgical package. The one or more functions can be non-compliant with one or more regulations of the geo-political location. In other embodiments, the one or more aspects can include one or more operations of the surgical instrument usable during a surgical procedure. For example, the one or more aspects can include the capacity to deliver one or more controlled substances, and the one or more controlled substances can be prohibited or controlled in the geo-political location. The geo-political location can be a customs checkpoint. In some variations, the instructions can be transmitted prior to crossing a geo-political border into the geo-political location.

In another embodiment, a non-transitory computer program product is provided. The non-transitory computer program product can store instructions which, when executed by at least one data processor forming part of at least one computing system, cause the at least one data processor to implement operations. The operations can include receiving, by a surgical system and from a remote server, data characterizing a geo-political location of the surgical system, and transmitting instructions, based on the received data, to the surgical package to selectively alter one or more aspects of the surgical package. The surgical system can include a manufacturer-sealed, sterile surgical package and a surgical instrument contained within the manufacturer-sealed, sterile surgical package.

In another embodiment, a surgical system is provided. The surgical system can include a manufacturer-sealed sterile surgical package containing a surgical instrument, at least one data processor, at least one memory storing instructions configured to cause the at least one data processor to perform operations. The operations can include receive, from a remote server, data characterizing one or more aspects of a surgical procedure involving the surgical instrument, determine a level of compatibility of the surgical instrument with each of the one or more aspects of the surgical procedure, and provide the determined level of compatibility.

The surgical system can vary in a number of ways. For example, the one or more aspects of the surgical procedure can include one or more surgical instruments, surgical accessories, and surgical techniques. The operations can include transmit a notification of incompatibility between the surgical system and the one or more aspects when the determined level of compatibility is below a compatibility threshold. In some aspects, the notification can be transmitted via a display located on the surgical system. In other aspects, the notification can be transmitted electronically to the remote server. In further aspects, the notification can include a list of the one or more aspects of the surgical procedure with which the level of compatibility is below the compatibility threshold. In still further aspects, the operations can include disable one or more features of the surgical system when the determined level of compatibility is below a compatibility threshold. For example, the providing can occur prior to breach of the manufacturer-sealed sterile surgical package containing a surgical instrument.

In another embodiment, a method is provided. The method can include receiving, by a surgical system and from a remote server, data characterizing one or more aspects of a surgical procedure involving the surgical system, determining a level of compatibility of the surgical system with each of the one or more aspects of the surgical procedure, and providing the determined level of compatibility.

The method can vary in a number of ways. For example, the one or more aspects of the surgical procedure include one or more surgical instruments, surgical accessories, and surgical techniques. The method can further include transmitting a notification of incompatibility between the surgical system and the one or more aspects when the determined level of compatibility is below a compatibility threshold. In some aspects, the notification can be transmitted via a display located on the surgical system. In other aspects, the notification can be transmitted electronically to the remote server. In further aspects, the notification can include a list of the one or more aspects of the surgical procedure with which the level of compatibility is below the compatibility threshold. In still further aspects, the method can include disabling one or more features of the surgical system when the determined level of compatibility is below a compatibility threshold. For example, the surgical system can include a manufacturer-sealed sterile surgical package containing a surgical instrument. In some variations, the providing can occur prior to breach of the manufacturer-sealed sterile surgical package containing a surgical instrument.

In another embodiment, a non-transitory computer program product is provided. The non-transitory computer program product can store instructions which, when executed by at least one data processor forming part of at least one computing system, cause the at least one data processor to implement operations. The operations can include receiving, by a surgical system and from a remote server, data characterizing one or more aspects of a surgical procedure involving the surgical system, determining a level of compatibility of the surgical system with each of the one or more aspects of the surgical procedure, and providing the determined level of compatibility.

In another embodiment, packaging is provided. The packaging can include a manufacturer-sealed, sterile surgical package, a surgical instrument stored within the manufacturer-sealed, sterile package, and at least one secondary-use package contained within the manufacturer-sealed, sterile surgical package and configured to receive and sterilely contain one or more components of the surgical instrument following the surgical procedure. The at least one secondary-use package can include at least one authenticator configured to selectively allow access to the at least one secondary-use package.

The method can vary in a number of ways. For example, the authenticator can be a watermark that is invisible to the human eye, and the at least one secondary-use package can be configured to open upon detection of the watermark. In other aspects, the authenticator can be at least one RFID chip embedded within the manufacturer-sealed, sterile surgical package. The at least one secondary-use package can be configured to open upon detection of the at least one RFID chip. In another embodiment, the authenticator can be a timer that is configured to prevent access to the at least one secondary-use package until a predetermined time period has elapsed. In other aspects, the authenticator can be at least one scannable marker. The at least one scannable marker can be configured to grant access to the at least one secondary-use package after being scanned. For example, at least one of the manufacturer-sealed, sterile surgical package and the at least one secondary-use package can include a biodegradable material. For example, at least one of the manufacturer-sealed, sterile surgical package and the at least one secondary-use package can include a ultraviolet-sensitive material that is configured to darken after exposure to ultraviolet light. For example, at least one of the manufacturer-sealed, sterile surgical package and the at least one secondary-use package includes at least one self-compaction feature to assist in the breakdown of the at least one of the manufacturer-sealed, sterile surgical package and the at least one secondary-use package. In some aspects, the at least one compaction feature can include two or more separable interlocking panels.

In another embodiment, a method is provided. The method can include accessing a surgical instrument stored within a manufacturer-sealed surgical package, performing at least one surgical operation with the accessed surgical instrument, storing at least one component within at least one secondary-use package contained within the manufacturer-sealed surgical package, and shipping the retained one or more components in the at least one secondary-use package following the at least one surgical operation. The secondary-use package can be shaped to retain one or more components of the surgical instrument.

The method can vary in a number of ways. For example, the method can include authenticating at least one authenticator located on the at least one secondary-use package to grant access to an interior thereof. In some aspects, the authenticator can be a watermark that is invisible to the human eye, and the at least one secondary-use package can be configured to open upon detection of the watermark. In other aspects, the authenticator can be at least one RFID chip embedded within the manufacturer-sealed, sterile surgical package. The at least one secondary-use package can be configured to open upon detection of the at least one RFID chip. In further aspects, the authenticator can be a timer that is configured to prevent access to the at least one secondary-use package until a predetermined time period has elapsed. In still further aspects, the authenticator can be at least one scannable marker. The at least one scannable marker can be configured to grant access to the at least one secondary-use package after being scanned.

### BRIEF DESCRIPTION OF DRAWINGS

The present invention is described by way of reference to the accompanying figures which are as follows:

FIG. 1 is a top view of a passive RFID tag, according to an embodiment;

FIG. 2 is a side schematic view of a smart packaging system containing a surgical instrument according to an embodiment;

FIG. 3A is a diagram of a smart packaging system in electronic communication with a beacon and a HUB in an operating room, according to an embodiment;

FIG. 3B is a diagram of a smart packaging system in electronic communication with a beacon and a HUB in an operating room, according to an embodiment;

FIG. 4 is a diagram of a supply chain for a smart packaging system including a surgical instrument, and the kinds of information exchanged to and from the smart packaging system at various points along the supply chain, according to an embodiment;

FIG. 5 is a side schematic view of a smart packaging system containing a surgical instrument and two indicators according to an embodiment;

FIG. 6 is a side schematic view of an indicator of FIG. 5 having radiation-sensitive ink before and after exposure to radiation;

FIG. 7 is a side schematic view of an indicator of FIG. 5 having a balloon before and after exposure to a change in pressure;

FIG. 8 is a side schematic view of a smart packaging system containing a surgical instrument having a killswitch, according to an embodiment;

FIG. 9 is a side schematic view of the smart packaging system of FIG. 8 after the killswitch has activated, according to a first variation;

FIG. 10 is a side schematic of the smart packaging system of FIG. 8 after the killswitch has activated, according to a second variation;

FIG. 11A is a perspective view of a smart packaging system including a tracker, according to an embodiment;

FIG. 11B is a top view of the smart packaging system of FIG. 11A;

FIG. 12A is a schematic of the components of the tracker of FIG. 11A, including a switch in a first position;

FIG. 12B is a schematic of the switch of FIG. 12A in a second position;

FIG. 13 is a perspective view of a nestable smart packaging system, according to an embodiment;

FIG. 14 is a diagram showing waste bins for receiving different kinds of waste according to a waste management plan;

FIG. 15 is a perspective view of a PCB and a pry bar configured to remove the PCB from a smart packaging system, according to some embodiments;

FIG. 16 is a schematic view of a smart packaging system and its components being marked according to a disposal plan, according to some embodiments;

FIG. 17 is a diagram of a recommendation process, according to an embodiment;

FIG. 18 is a simplified side view of a patient's stomach with scar tissue and markers of a prior surgery;

FIG. 19 is a perspective view of the stomach of FIG. 18;

FIG. 20 is a top view of a smart packaging system including a surgical instrument and surgical accessories, according to a first variation of an embodiment;

FIG. 21 is a top view of a smart packaging system including a surgical instrument and surgical accessories, according to a second variation of the embodiment of FIG. 20;

FIG. 22 is a perspective view of a smart packaging system including a surgical instrument and surgical accessories and having tiered trays, according to an embodiment;

FIG. 23A is a side view of packaging of a smart packaging system in a nested configuration, according to a variation of some embodiments;

FIG. 23B is a side view of the packaging of FIG. 23A in a collapsed configuration;

FIG. 24A is a side view of packaging of smart packaging systems, according to a variation of some embodiments;

FIG. 24B is a top view of the packaging of FIG. 24A in a nested configuration;

FIG. 25 is a top view of a packaging with a plurality of nesting locations therein, according to an embodiment; and

FIG. 26 is a chart depicting a watermark authentication process, according to an embodiment.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Smart surgical devices, systems, and methods are provided. The use of smart devices, systems, and methods can generally enable the storing, sharing, and utilization of information throughout a supply chain, as well as to assist in the management of various systems, procedures, and aspects of healthcare facilities in which they are used. At all times, information directed to aspects of the supply chain and of healthcare facilities can be logged, monitored, and reviewed in order to adjust facets of the supply chain and/or healthcare facilities in real time. Additionally, analysis of the compiled information can be used in order to minimize or avoid pitfalls and issues associated with the supply chain and/or products flowing through the supply chain, as well as to minimize or avoid pitfalls and issues associated with the coordination of surgical procedures and associated devices at healthcare facilities. Overall, smart devices, systems, and methods can improve the efficiency of the supply chain through the management of information associated with products flowing through that supply chain, and they can improve the efficiency of the healthcare facilities in which they are deployed for use in surgical procedures. While the specific types of smart devices, systems, and methods can vary, in some aspects the packaging of the products themselves can be leveraged in order to consistently track, monitor, and record information associated with the products. Further, tracking devices (e.g., scanners, beacons, etc.) and/or a centralized computer management system can be employed as part of the smart systems and devices.

The use of smart devices, systems, and methods can also impact the operations of a healthcare provider, such as during a surgery involving the smart surgical devices. Information can be recorded, stored, monitored, and acted upon before, during, and after a surgical procedure involving the smart surgical device in order to improve the performance and to minimize the operational risks of the smart surgical device and of future smart surgical devices.

Smart devices, including smart packaging systems, can act on the information they receive to provide recommendations, warnings, guidelines, and other information to various personnel associated with healthcare facilities. This provided information can contemplate all aspects of daily procedures at healthcare facilities, including scheduling, logistics, coordination of surgical procedures, recommended handling instructions during specific surgical procedures, and more. Moreover, the smart devices and smart packaging systems themselves can be designed to include various physical aspects intended to assist personnel in acting upon the provided information.

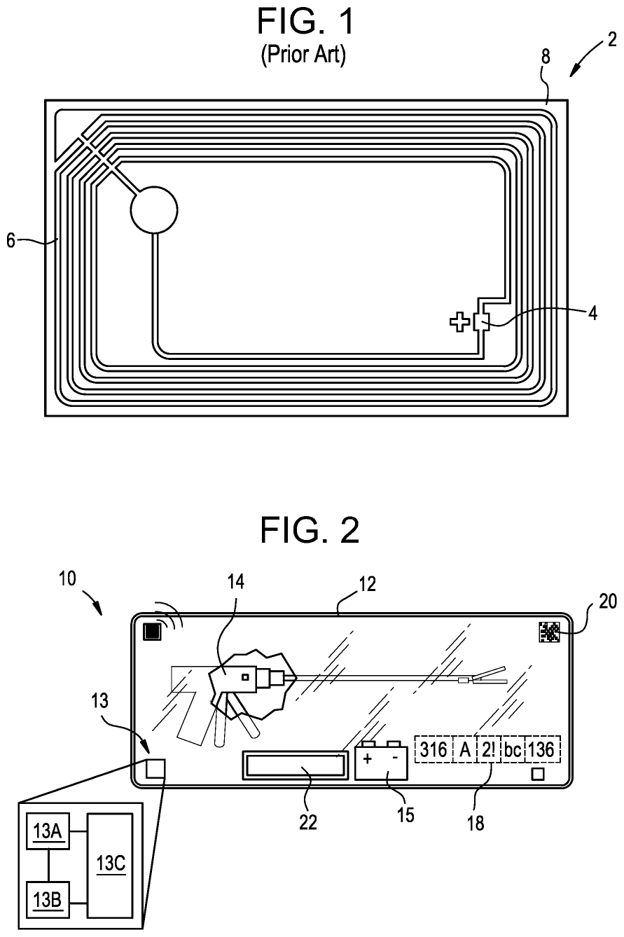

As indicated above, in some aspects the smart devices, systems, and methods can utilize smart packaging systems, which can be included on an outer packaging containing one or more surgical instruments. In an exemplary embodiment, the outer packaging can include a power source, one or more radio frequency identification (“RFID”) tags, and one or more sensors capable of measuring environmental aspects. The one or more RFID tags can take on various forms and may generally include passive and active RFID tags. Passive RFID tags can include an RFID chip (or integrated circuit, “IC”), an antenna, and a substrate. The IC contains a logical control unit, memory, and transceiver, which can be used for decoding, decrypting, and error checking. The antenna is used to receive/transmit information, such as electronic data, to and from an external electronic system with its own reception and/or transmission capabilities (e.g., a reader). The substrate holds the chip and antenna together and provides the RFID tag with structure. Passive RFID tags can receive power in the form of electromagnetic energy (e.g., radio energy) transmitted by a reader and received by the antenna of the passive RFID tag. An example of a passive RFID tag 30 can be seen in FIG. 1. The passive RFID tag 2 includes an IC 4 electronically coupled to an antenna 6. The IC 4 and the antenna 6 are mounted on a substrate 8.

Active RFID tags may include components similar to those of passive RFID tags with the addition of a separate power source (e.g., an integrated battery). Further, in some variations, other kinds of sensors or chips, such as near field communication (NFC) sensors, may be used in addition to, or in place of, the one or more RFID tags.

In some variations, the outer packaging can include additional memory storage and/or one or more additional processors in electronic communication with the one or more RFID tags in order to increase the capabilities of the RFID tags. The smart packaging system can also include one or more of a display, such as an e-ink display, LCD display, touchscreen, or equivalent, and/or a readable medium, such as a barcode, QR code, or equivalent. In some variations, the smart package system can include data ports, such as USB-type ports. With some or all of these features, the smart packaging system can generally be capable of taking in data from external sources, such as computers, computer networks, and data received from its one or more sensors, and the smart packaging system can generally be capable of transmitting data and/or presenting information to computers, computer networks, users, scanning devices, RFID readers, and any device capable of receiving data from features of the smart packaging system.

The surgical instrument(s) and/or surgical components contained within the outer packaging can vary in both form and function. In some variations, the entire surgical instrument can be a “smart” instrument in which the status and operations of the surgical instrument can be monitored, recorded, and altered at any time. For example, if the surgical instrument is an endo-cutter configured to incise and staple tissue, an amount of torque applied by the jaws of the instrument can be monitored throughout a surgical procedure. If it is determined by the HUB that too much torque is being applied by a surgeon, a maximum torque limit can be imposed wirelessly on the instrument in real time so as to prevent the occurrence of an accident. Further, with such surgical instruments, if, during an operation involving the instrument, an accident does occur, details surrounding the accident can be recorded and stored either within the memory of the package or virtually on the HUB. This information can then be used to determine the source of the accident, and, for example, whether a recall should be issued for similarly-situated surgical instruments.

In other variations, sub-portions of the surgical instrument can be “smart,” while other portions of the instrument may be electronically isolated from the “smart” sub-portions such that the status and operations of the surgical instrument are not capable of being monitored, recorded, or altered whatsoever. For example, in the same endo-cutter described above, a jaw-driver sub-system may be “smart,” but a sub-system concerning articulation of a shaft of the endo-cutter may not be “smart.” Even though the shaft may be electronically articulable, it may not be in electronic communication with the HUB and/or the outer packaging, and it, accordingly, may be “hidden” from monitoring. These examples are exemplary only, and more concrete examples and variations are described below.

An example of a smart packaging system 10 is depicted in FIG. 2. The illustrated smart packaging system 10 includes a packaging 12 containing a surgical instrument 14 therein. The surgical instrument 14 can be any surgical instrument or surgical material, such as an endocutter as shown. The smart packaging system 10 can include a controller 13 that is configured to execute functions of the smart packaging system 10. The controller 13 can generally include a control unit 13A, a logic unit 13B, and a memory 13C that enable functionality of the controller 13. The smart packaging system 10 can also include an internal power source 15, such as a battery, that is capable of providing power to various components of the smart packaging system 10, including the controller 13, described herein. The packaging 12 can also include an RFID tag 16 configured to transmit and receive information from an external source. A serial number 18, which may or may not feature encrypted data, can be locating on the packaging 12 such that it can be read by a handler or a computer system. The packaging 12 can further include a scan-able or readable medium 20, such as a QR code or a barcode and a display 22, such as the kind mentioned previously (e.g., an e-Ink display). While not shown, the smart packaging system 10 can contain additional surgical accessories, components, and elements that may be disposed in a compartment separate from the surgical instrument 14. The surgical accessories, components, and elements can be used in conjunction with or separate from the surgical instrument 14. Together, these items can generally be referred to as the contents of the smart packaging system 10. For example, the surgical instrument 14 can be a surgical stapler, and the contents of the smart packaging system 10 can include staple cartridges that are compatible with the surgical stapler, which may be packaged within the smart packaging system 10 separate from the surgical stapler, such as in a sub-compartment of the packaging 12. Reference will be made to the smart packaging system 10 and its component parts for exemplary purposes only.

The smart packaging system 10 can receive and present information relevant to personnel who may interact with the smart packaging system 10 along the life cycle of the contained surgical instrument 14, as well as with other surgical instruments and products that interface with the contained surgical instrument 14. The kind of personnel and the relevance of the information will vary depending on where the surgical instrument 14 and the smart packaging system 10 are in their lifecycle. Following manufacture in a factory, the surgical instrument 14 will be packaged and prepared for delivery to eventually end up at a healthcare facility, such as a hospital or surgery center, to be used in a surgical procedure. To arrive at that the hospital or surgery center, the smart packaging system 10 will undergo a series of interactions with various personnel, including factory personnel, warehouse personnel, transit personnel, customs personnel, and hospital personnel. At each phase of its journey, the smart packaging system 10 can receive and transmit relevant information to personnel. Although the smart packaging system 10 can store a variety of information, the smart packaging system 10 can, based on context, selectively provide only relevant information.

Additionally, on its journey, the smart packaging system 10 may communicate with a central network, also called a HUB 50, which can monitor and coordinate information including and about the smart packaging system 10. The HUB 50 can be present along the smart packaging supply chain in the form of beacons 102 placed in key locations, such as a hospital operating room (OR). When a beacon 102 or other facet of the HUB 50 detects that the smart packaging system 10 is in close proximity, information can be exchanged between the smart packaging system 10 and the HUB 50, and information can be exchanged between the HUB 50 and various external networks.

Each beacon 102 can include a transceiver, a transponder, a power source, a processor, and a local memory. The beacons 102, generally can operate as an RFID reader in electronic communication with a central network, HUB 50. The beacons 102 can be in wired communication or in wireless communication, such as via infrared communication, radio communication, Wi-Fi communication, Bluetooth, etc. The RFID reader can be a passive reader that can receive signals transmitted by an active RFID tag, the RFID reader can be an active reader that can transmit interrogator signals and receive replies, such as authentication replies, from an RFID tag. During operation, the beacons 102 can receive transmitted data from the smart packaging system 10 characterizing information recorded by the smart packaging system 10 related it its experiences since manufacture. The beacons 102 can then transmit that received data to the HUB 50 where it can be stored, analyzed, and/or acted upon. The beacons 102 can also transmit information from the HUB 50 to a nearby smart packaging system 10.

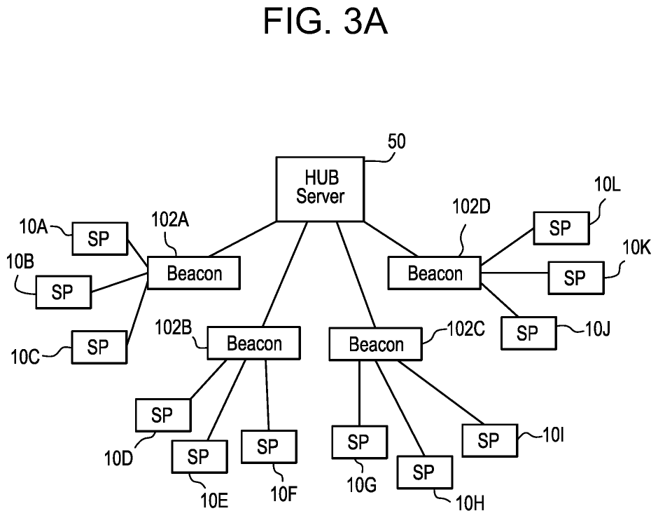

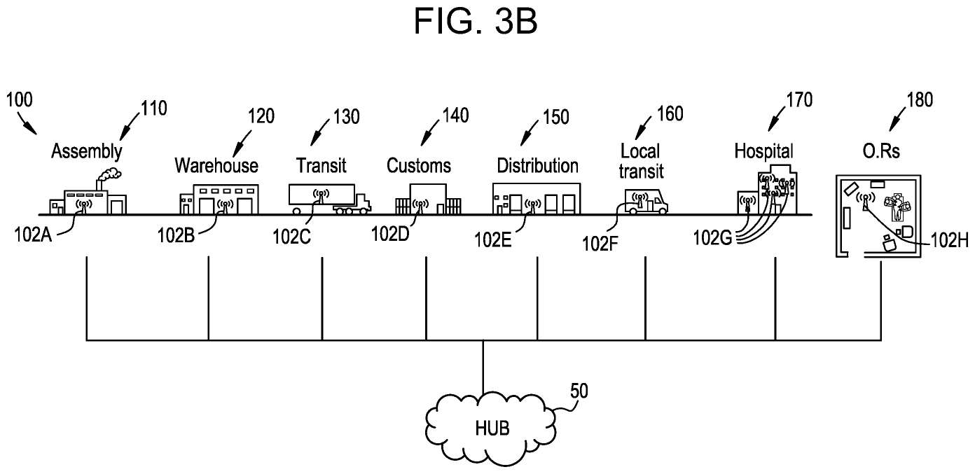

A non-limiting example of the exchange of information between the beacon 102 (or HUB 50, generally) and the smart packaging system 10 is shown in FIG. 3A and FIG. 3B In FIG. 3A, a chart is depicted that shows a general hierarchy structure between the HUB 50, the beacons 102 (individually represented as beacons 102A-102D), and the smart packaging system 10 or systems 10 (individually represented as smart packaging systems 10A-10L). The HUB 50 can be in communication with the beacons 102, which can be in communication with the smart packaging systems 10. In some variations, however, the smart packaging system 10 can transmit information directly to the HUB 50 and receive information directly from the HUB 50 using its own transmission and reception capabilities. In FIG. 3B, a chart is depicted showing an exemplary supply chain 100, and different contexts in which the smart packaging system 10 may communicate with the HUB 50 via one or more beacons 102. The interplay between the smart packaging systems 10 and the HUB 50, via the beacons 102, can be important depending upon the context in which the smart packaging system 10 is located. This context can change as the smart packaging system 10 moves from manufacture to transit to use through a supply chain.

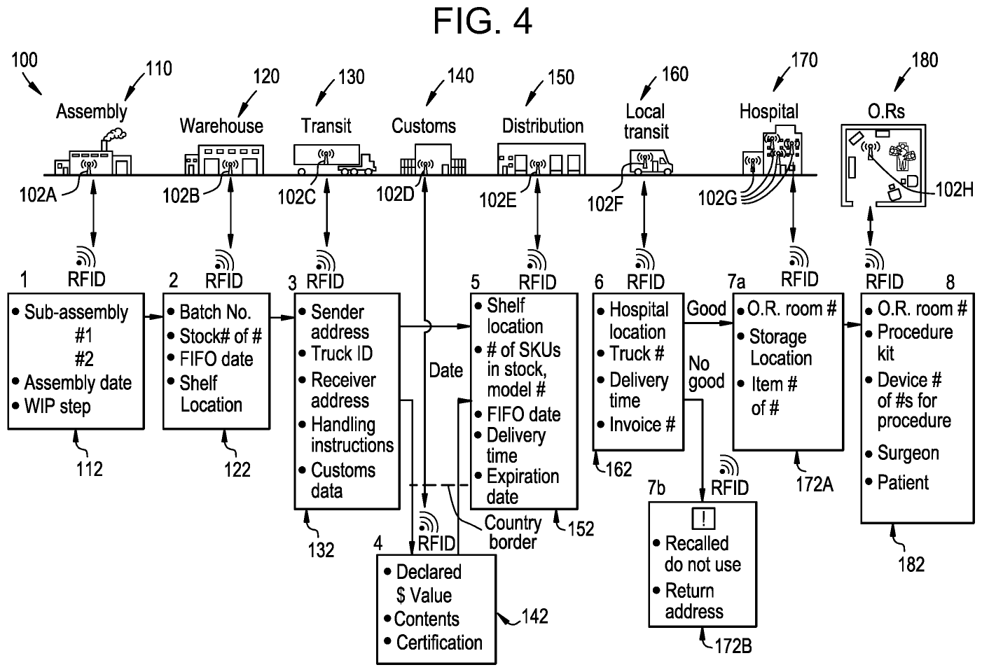

The supply chain 100 shown in FIG. 3B and in FIG. 4 is shown with simplified versions of key stages along the supply chain of a surgical device or system, making the depiction of the supply chain 100 illustrative and exemplary, rather than mandatory and necessary. In reality, actual supply chains may include these stages in a different order, or they may include additional stages, duplicate certain stages, or omit stages altogether.

The first depicted stage in the supply chain 100 is the assembly stage 120, which can include a beacon 102A to facilitate communication with the HUB 50. The assembly stage 120 represents manufacture of the surgical instrument 14. Information exchanged at the assembly stage 120 can include assembly information 112 pertaining to the surgical instrument 14 itself, including its performance metrics, manufacture specifications, tolerances, safety and usage information, etc. The assembly information 112 can also include, for example, sub-assembly information pertaining to one or more sub-assemblies that are compatible with the surgical instrument. Additionally, the assembly information 112 can include work-in-progress (WIP) step and assembly date of the surgical instrument.

The second depicted stage in the supply chain 100 is the warehouse stage 110, which can include a beacon 102B. The warehouse stage 110 represents storage of the smart packaging system 10 prior to shipment thereof. Information exchanged at the warehouse stage 110 can include warehouse information 122, such as batch number, stock number and count, FIFO date, and shelf location so that the smart packaging system 10 can be tracked and located within the warehouse itself.

The third depicted stage in the supply chain 100 is the transit stage 130, which can include a beacon 102C. The transit stage 130 represents transit of the smart packaging system 10 from the warehouse to end up at a distribution center. Information exchanged at the transit stage 130 can include transit information 132, such as sender address, truck ID, receiver address, handling instructions, and customs data.

After the third depicted stage, the smart packaging system 10 may optionally be shipped across geopolitical borders, including to international locations, before ending up at a distribution center. The fourth depicted stage is the customs stage 140, which can include a beacon 102D, and it represents those occurrences when the smart packaging system 10 must cross a geopolitical border. Information exchanged at the customs stage can include customs information 142, such as declared value, contents, and certification.

If the smart packaging system 10 is not be shipped internationally, the smart packaging system can proceed from the transit stage 130 directly to the distribution stage 150. If the smart packaging system 10 is shipped internationally, it can proceed to the distribution stage 150 following the customs stage 140. The distribution stage 150 represents a warehouse or distribution center, which can include a beacon 102E that receives the smart packaging system before it is sent to its ultimate destination, such as a hospital. Information exchanged at the distribution stage 150 can include distribution information 152, such as shelf location, number of specific stock keeping units (SKUs) in stock, model number, FIFO date, delivery time, and expiration date.

The sixth depicted stage in the supply chain 100 is the local transit stage 160, which can include a beacon 102F. The local transit stage 160 represents the transit required to get the smart packaging system 10 to its ultimate destination. Information exchanged at the local transit stage 160 can include local transit information 162, such as hospital or surgery center location, truck number, delivery time, handling instructions, and invoice number. During local transit, if any aspect of the smart packaging system has been recalled, the information exchanged can include, for example, a return address, and the smart packaging system 10 can be recalled and returned for assessment and/or disposal.

The seventh depicted stage in the supply chain 100 is the hospital stage 170, which can include one or multiple beacons 102G. The hospital stage 170 represents the hospital or surgery center in which the surgical instrument 14 will be used during a surgical procedure. If local transit is able to deliver the smart packaging system 10, local transit will deliver the smart packaging system 10 to the hospital where it can be stored within the hospital until needed. Information exchanged at the hospital stage can include hospital information 172A, such as operating room (OR) number, storage location, and item count. If local transit is not able to deliver the smart packaging system 10, such as for a recall, the smart packaging system 10 can exchange recall information 172B, such as a return address for the smart packaging system, and a warning with information related to the recall.

The eighth depicted stage in the supply chain 100 is the OR stage 180, which can include a beacon 102H. The OR stage 180 represents an operating room that will use the surgical instrument 14 that is part of the smart packaging system 10. Information exchanged at the OR stage 180 can include OR information 182, such as OR room number, procedure kit details, device number in the total devices needed for the procedure, surgeon information, and patient information.

Information described at each of the stages in the supply chain 100 is exemplary only and does not represent an exhaustive list of the information that can be exchanged at a given stage.

Generally, the smart packaging system 10 can provide a user (e.g., transportation worker, customs officer, surgeon, warehouse personnel, etc.) with information concerning itself. The information can be conveyed by the smart packaging system 10 in a number of ways, as will be described below, and the information conveyed can be dependent on a number of factors, including: composition, contents, present location, destination, identity of the user, authorization level, functional capabilities, external stimuli, and more.

In some embodiments, the smart packaging system 10 is configured to store its intended destination (e.g., a specific hospital), and to document and store a historic record of its travels as it progresses toward its intended destination. Additionally, the smart packaging system 10 can be configured to determine its location within the pathway to its intended destination.

The smart packaging system 10 can locate and monitor aspects and components of the smart packaging system 10, such as the surgical instrument 14, surgical accessories and contents contained therein, and other elements within an operating room. This information can be broadcasted or otherwise provided as needed. For example, as items enter an operating room containing a smart packaging system 10, they can be added to a database containing a list of components present in the operating room in real time. The list can be stored on the smart packaging system 10 or on a device in electronic communication with the smart packaging system 10, the HUB 50, or another similar system. If the smart packaging system 10 enters an operating room already containing items, those items can be added to the list upon first detection. As items on the list leave the operating room or are disposed of, they can be removed from the list.

Items can be added to the list by manual input or by scanning (e.g., via a barcode or QR code or the like, or by remote scanning by an electronic system, such as the HUB 50), or by any combination of the methods described herein. When items are added to the list using manual input, healthcare personnel can enter information characterizing the items present in an operating room. As the items are disposed of or otherwise removed from the operating room, the items can be manually deleted from the list. When scanning is the method of input, a variety of methods can be used. These methods can include manual scanning, active wireless transmission by capable devices, direct detection, geo-fencing, BLE beacons, and by surgical step.

Manual scanning can also involve healthcare personnel, but instead of personnel manually entering attributes of items as they enter an operating room, the item can be scanned using any described methods, and attributes of the scanned item can auto-populate the list. For example, by scanning a barcode of a smart packaging system 10, attributes of the smart packaging system 10, such as its contents (e.g., surgical instrument 14, surgical accessories, etc.) and related details, operative capabilities of the contents, and more can be obtained from the barcode and added to the list.

Active wireless transmission is one kind of technique for object monitoring. Active wireless transmission can operate between capable devices to transmit information between those devices. A polling device, such as the HUB 50 or a smart packaging system 10, can transmit a wireless poll or tally. Capable devices within the range of the wireless poll or tally can respond to the wireless poll or tally by transmitting their own signal toward the polling device. Based on the signal transmitted by the polled devices, both a direction and distance to each of the detected devices can be determined. With this information, the polling device can further determine whether the detected device or devices is within an operating room or area of interest. Devices determined to be within the operating room can be added to the list.

This methodology can even be used to detect devices which are not currently within an area of interest, such as the operating room, but that should be within that area of interest. For example, if a polling device transmits a signal and detects a device that is within range but outside the operating room, and the polling device knows that the detected device should be in the operating room, a notification can be provided informing a user of this information. Additionally, the direction and distance to the detected device or devices can be provided to a user so that the user can locate the detected device or devices. This information could be provided on the display 22 of the smart packaging system 10, on a display of the polling device, or on a monitor in electronic communication with transmitting device, such as a monitor located within the operating room. Further, the detected device or devices could be requested via transmission to provide secondary local feedback to enable location, i.e., the detected device or devices send out their own transmission to receive information about their current location, proximate devices, etc., and then that information can be provided to the initial polling device. The detected devices could also be made to play an audio signal if capable.

Direct detection of an aspect of the device or component is another kind of technique for object monitoring. If a sought device has certain physical properties, detection can take advantage of those physical properties to locate the sought device. For example, if the sought device is known to be metal, that metal can be detected via impedance or capacitance field monitoring to locate the sought device in a scanned area.

An active global geo-fence is another kind of technique for object monitoring. An active global geo-fence can be assigned to a specific geo-physical location, and all objects passing through that fence can be tracked. A combination of systems could be combined to monitor objects passing through this fence, such that the combination of systems could detect objects in a combined device or as separated elements. This kind of monitoring could function in a number of ways, such as relying on a specific visual indicator present on an object to constantly track that object. For example, a package of surgical needles could include a specific yellow and black stripe pattern. A camera included in the systems could be relied on to visually monitor an area for that specific yellow and black strip pattern, even at multiple angles. When the pattern is detected, the object can be added to the list of items known to be present within the geo-fence. One example of a detectable object includes some surgical sponges, which can have integrated RF transmitters. The surgeon, or other personnel, can use a corresponding device, such as a wand, to determine whether the sponges were left behind following a surgical procedure. One advantage provided by the geo-fence would include automatic tracking and detection of such sponges to insure the removal of the sponges without requiring active detection by the surgeon or other personnel.

Bluetooth low energy (“BLE”) beacons can be employed for another kind of technique for object monitoring. The BLE beacons can repeatedly transmit a signal that other devices can detect, such as via a radio signal. The signal can comprise an encoded message of letters and numbers transmitted on short intervals, which can be used for specific transmissions and to encode specific messages and data. The BLE beacons can be used to register products currently located in an operating room, and they can also be used to communicate status changes of detected objects and products. Once the objects enter an operating room, the initial detection by the BLE beacon can add that object to a database list. Further, if a status change occurs, that information can be communicated to other networks. For example, if a smart packaging system 10 is opened, the beacon can change its message to indicate that a given product is currently in use in an operating room. A signal can be transmitted to the HUB 50 to mark that opened smart packaging system 10 off a stock tally for the overall healthcare facility to indicate that the opened smart packaging system 10 is no longer available in stock. In another example, if an object leaves the operating room, a BLE beacon can transmit a message to indicate whether that object is returning to a central storage or that the object has been consumed and is now entering a waste stream.

Tracking objects by surgical step is another kind of technique for object monitoring. Tracking objects by surgical step may need to rely on previously-described techniques, such as geo-fencing, in order to provide additional information used for object monitoring. For example, sub-areas within an operating room can be designated (e.g., a surgical field) using geo-fencing. Objects known to be needed for a surgical procedure, yet which have not been detected entering a geo-fenced surgical field, can provide an indication that a certain step in the surgical procedure has not yet occurred.

Multiple communicating devices within an operating room setting can be disruptive and lead to interference between signals transmitted by those communicating devices. A number of techniques can be employed to minimize disruption. For example, the smart packaging system 10 can contain one or more methods to alter a bandwidth of its transmissions, as well as multiple methods of communication. Some of the methods of communication can rely on the internal power source 15 of the smart packaging system 10. Some methods can derive power from an external source, such as an external electro-magnetic field, such as with near field communication (NFC), or with an external wired connection, such as via a USB cable or the like.

The smart packaging system 10 can rely on RFID tags to communicate with other devices, but if RFID becomes unavailable, the smart packaging system can rely on secondary means of communication. For example, NFC and RFID could be combined such that, with RFID, power is obtained from an external field and broadcasting only occurs when the system is prompted. With the NFC, an antenna can be used, and upon activation, other systems, such as WIFI and battery systems, can be deactivated to save power.

Other techniques to minimize interference can vary. For example, surgical carts (described herein) can be smart-enabled, and when a smart device is placed upon a smart cart, the smart device can detect the smart cart and can relinquish communication capabilities to the smart cart, i.e., the smart cart can maintain a list of devices placed upon it and can handle all location requests transmitted to devices on the list. For example, if a smart packaging system 10 is opened, location-related communications can be deactivated. For example, location-related communications can be disabled based upon a current location of the smart device. If the smart device is being shipped in transit, location-related request capabilities can be deactivated to conserve power. For example, a smart device can broadcast a limited number of times for a given event. If a device is programmed to broadcast a change in its location during movement of that device, the device can instead be limited, at least in some scenarios, to provide less frequent broadcasts. For example, smart devices can operate in a tiered system, such that all transmissions by smart devices are sent to a central point, such as the HUB 50, and the HUB 50 routes the transmissions to their proper receiver while prioritizing and coordinating the transmissions to minimize interference.

In some embodiments, the smart packaging system 10 can document information related to aspects of its assembly and manufacturing, and the smart packaging system 10 can store and provide that information to various personnel interfacing with the smart packaging system 10, as well as to make certain decisions based in part on the stored information. The information stored can vary, and can include information related to aspects that ultimately impact use of the smart packaging system 10 and its contents, including the surgical equipment contained therein (e.g., surgical instrument 14). For example, the stored information can include: information related to calibrations and specific settings of the smart packaging system 10, which can adjust operation of the smart packaging system 10 based on combinations of settings and equipment interactions; information related to manufacturing processes and manufacturing materials of the smart packaging system 10; and information related to special instructions for use of the smart packaging system 10, as well as limitations of operations, such as adverse reactions with other equipment.

When the information relates to calibrations and specific settings of the smart packaging system 10, that information can specifically include information pertaining to calibrations of the smart packaging system 10 and its contents that adjust operation of the contents (e.g., the surgical instrument 14) based on other devices paired with or used with the contents. This can be accomplished by locating or determining key aspects of the smart packaging system 10 and its contents in relation to various combinations of accessories and equipment combined with or grouped with the smart packaging system 10 and its contents. For example, a surgical instrument 14 may be operable with a wide range of surgical needles, each of which can be tagged with an RFID tag (e.g., similar to RFID tag 16) or equivalent. When the surgical instrument is paired with a certain tagged surgical needle, calibrations of the surgical instrument can be adjusted based on that pairing. The information required to make the adjustment can be coordinated via the HUB 50, or the surgical instrument 14 can be programmed to recognize the consequences of a pairing with that certain kind of tagged surgical needle.

Locating or determining key aspects of the smart packaging system 10 and its contents in relation to various combinations can improve the traceability of constituent components that make up the smart packaging system 10. This traceability can benefit sustainability efforts, safety efforts, and more. The smart packaging system 10 can store a log or listing of the constituent components of the smart packaging system 10, such as various needles, blades, batteries, sensors, housings, etc. Separable components can be identified as special elements that must be accounted for following a surgical procedure or other procedure. Additionally, the manufacturing origin of each component of the smart packaging system 10 can be contained in the metadata of the smart packaging system 10. This information and its uses are elaborated on in more detail below.

When the information relates to manufacturing processes and materials, that information can specifically include information related to manufacturing processes of the components of the smart packaging system 10, as well as component composition.

Manufacturing tracking information can include information related to the procedures that components of the smart packaging system 10 undergo from assembly to shipping. For example, once the components are assembled, tracking information can record when the components are compiled into a smart packaging system 10. The smart packaging system 10 can be marked with a base serial number, as well as any other special identification information necessary. The smart packaging system 10 can also be marked with one or more additional serial numbers related to specific components contained therein, as well as being marked with additional data to assist in post-assembly procedures, such as information helpful to assess the smart packaging system 10 following a potential recall. The smart packaging system 10 can also be marked with information related to manufacturing data and site.

Manufacturing tracking information can also include sterilization count and procedures, and related sterilization information, including data of sterilization.

Manufacturing tracking information can also include information related to the entry of the smart packaging system 10 in bulk packing/shipping procedures. For example, in variations of the smart packaging system 10 that rely on a smart display 22, the smart display 22 can be configured to display information relevant to a current status of the smart packaging system 10, and, if not current status is known or discernable by the smart packaging system 10, the display 22 can default to the last known location. The display 22 can further be configured to display information related to contact information of the manufacturer.

Component composition information can include information relevant to the composition of a given component of the smart packaging system 10. This information can include any noteworthy materials of the component, or in the case where two alternative compositions may be employed, the specific composition of that component such that there no ambiguity. For example, the smart packaging system 10 can include a surgical instrument 14 in the form of an endocutter. Certain endocutters can include a channel retainer that can be either plastic or aluminum, which can impact a decontamination processes of the endocutter, as well as affecting disposal procedures, among other reasons. The component composition information can specifically include an indication as to whether the channel retainer is plastic or aluminum to clarify the ambiguity surrounding decontamination procedures of the endocutter.

In another example, component composition information can communicate whether radioactive needles were included in a smart packaging system 10. Such an inclusion could impact labeling requirements and travel restrictions of the smart packaging system 10 as applied to certain jurisdictions. The component composition information can include information related to other regulated components as well, which may face restrictions when shipped internationally. For example, cobalt has certain regulatory restrictions in Europe, and that information would be noteworthy in a European regulatory context.

Generally, the smart packaging system 10 can be aware of its historic and future states, as well as its intended usage, throughout its lifecycle. In essence, the smart packaging system 10 can possess an awareness of itself during its lifecycle. This awareness can result in the smart packaging system knowing: 1) its location within a facility supply chain; 2) when it has been received at a medical facility; 3) when it enters a stockroom or storage at the medical facility; 4) when it is requested for usage in a medical procedure; and 5) when it enters an operating room for the requested medical procedure. Each of these aspects of awareness will be further described.

Locations that trigger different labels or transmittals of information, generally, can include: warehouse, storage, transit, sterilization, supplier distribution center, mode of transit, customs, regional distribution center, local delivery, healthcare facility receiving, store room, operating room, repackaging in an operating room, healthcare facility cleaning/sorting, disposal, reuse, return shipping, etc.

The smart packaging system 10 can be aware of its location within a facility supply chain. Specifically, the smart packaging system 10 can be aware of virtual kits, as described herein. The smart packaging system 10 can also identify connectivity components to enable interfacing with various facility equipment, procedures, etc. The smart packaging system 10 can also identify missing capital equipment within an operating room. For example, if a certain software is out of date, it can be flagged by the smart packaging system 10 such as through a notification on the display 22 or via a transmission by the smart packaging system 10 to a device in electronic communication with the smart packaging system 10. The smart packaging system 10 can identify attempted communication with incompatible systems. The smart packaging system 10 can relay information to other devices identifying the incompatible system in order to prevent communication attempts with those other devices by the identified system. This information can also be relayed to the HUB 50, which can inform the proper user.

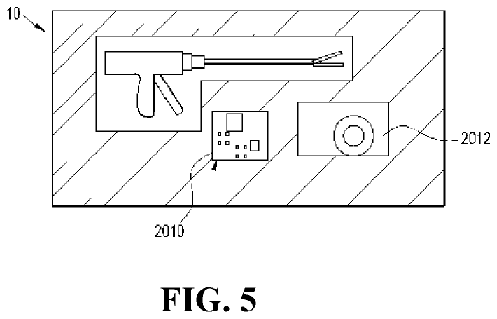

The smart packaging system can include at least two separate visual indicators of sterility, seen, for example, in FIG. 5, which depicts the smart packaging system 10 according to another variation. The smart packaging system 10 can include a first indicator 2011 and a second indicator 2012. The indicators 2011, 2012 can communicate a change in sterility, such as loss of sterility within the smart packaging system 10. Each indicator 2011, 2012 can be an element that changes in appearance, either changing color or changing from invisible to visible. A first indicator 2011 can indicate whether the smart packaging system 10 has been sterilized adequately. A second indicator 2012 can indicate whether sterility has been lost. Each indicator 2011, 2012 can be initially triggered by a sterilization event, but the second indicator 2012 can be triggered in the event that sterility is lost, while the first indicator 2011 remains unchanged.

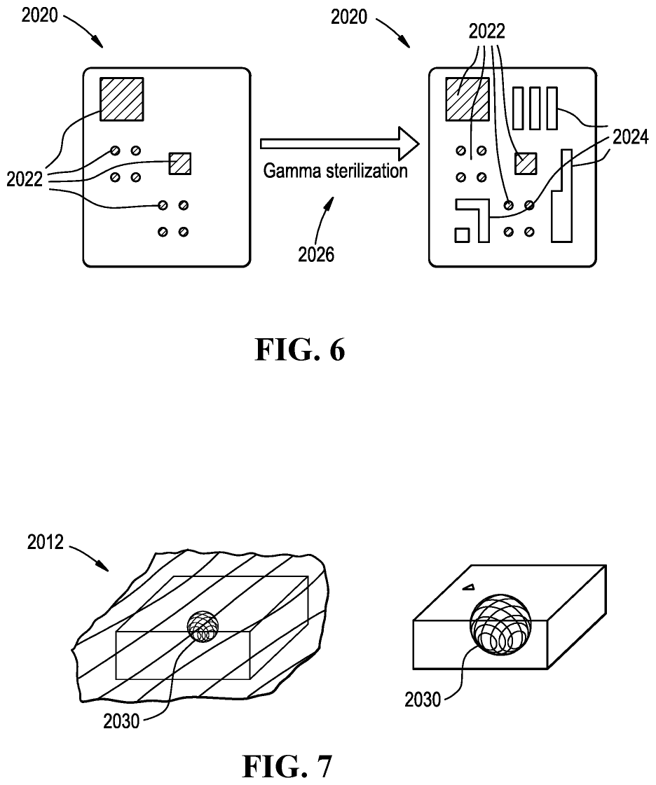

The first indicator 2011, seen also in FIG. 6, can include a portion of a code 2020 on the smart packaging system 10, such as a QR code or barcode. The code 2020 can be printed in a first portion 2022 in a standard ink to form an incomplete code. A second type of ink can be used for a second portion 2024 of the code 2020. The ink can darken when exposed to gamma radiation, the preferred method of sterilization of the smart packaging system 10, thereby completing the code 2020 and facilitating scanning thereof. Unless the smart packaging system 10 is sterilized via gamma radiation 2026, the second portion 2024 of the code 2020 never darkens and the code 2020 cannot be scanned, thereby indicating that the smart packaging system 10 was not properly sterilized and should not be used.

For the second indicator 2012, seen in FIG. 7, for example, the packaging process can include a step to trap inert gas or vacuum within a balloon 2030 or similar device stored in the smart packaging system 10 at a pressure (P1) different than atmospheric pressure. The balloon 2030 is placed within the sealed smart packaging system 10, and an interior of the balloon 2030 is held at atmospheric pressure (P2). If the outer seal is breached such that that the P2 is now equal to P1 at atmospheric pressure, the balloon 2030 or similar device would change in size as a result, thereby indicating that the smart packaging system was breached and the sterility is lost.

An alternative second indicator (not shown) could involve a double-walled balloon located within the smart packaging system 10. The inner balloon contains a volume under a first pressure P1 and the outer balloon contains the inner balloon and is held at a second pressure P2 between the outer and inner balloons. The outer balloon is made from a thin material and bursts upon sterilization, releasing its contents into the smart packaging system 10 and freeing the inner balloon. This results in an increase in pressure within the smart packaging system (assuming P2 was greater than atmospheric pressure). The inner balloon is still held at P1, and upon breach of the smart packaging system 10, the pressure will drop to atmospheric pressure, thereby altering the shape of the inner balloon and indicating a breach in sterility. These indicators 2011, 2012 can assist in avoiding the reuse of packaging and can assist in protecting against counterfeiting.

Turning back to the supply chain, the smart packaging system 10 can be aware of when it is in a warehouse and when it is in transit, and it can transmit information, via the display 22 or some other means described herein, as needed. This information can include serial number, batch number, manufacturing date, country mode code, means of transit, etc. In a warehouse, the transmitted information can include UDI, batch number, and other information relevant to warehouse operations. When the smart packaging system 10 determines it is being loaded into transit, the information can change to include address, handling instructions, etc. The smart packaging system 10 can display shipping identification information as well. When it is received by a distributor, the information transmitted can include expiration date, bin number, location, count, etc. The smart packaging system 10 can also display information relevant to a current or future geo-political location, such as information relevant to regulatory information of that location. For example, if the smart packaging system 10 contains lithium-ion batteries, the smart packaging system 10 can display a warning explaining that lithium-ion batteries cannot be transported on a commercial flight. When the smart packaging system 10 is with a supplier, the transmitted information can include first-in first-out information, batch number, a unique identifier, build aspects, etc. The supplier can be interested in where the smart packaging system is going, how many will be shipped, where it should be stored, and other information. When the smart packaging system 10 is at customs, the transmitted information can include value, destination, source, generic title of the product, hazardous materials warnings and descriptions, fragility, perishable, liquid, etc. When the smart packaging system 10 is on its way to a medical facility, the information transmitted can include address of the facility, invoice, list of items in the shipment, purchase order number, handling requirements (e.g., refrigeration, etc.).

The smart packaging system 10 can be aware of when it has been received at a healthcare facility, for example, upon an initial scan by the HUB 50, upon an initial scan by facility personnel, upon manual or remote activation, upon determination of a present geolocation, upon detection of compatible systems indicative of that healthcare facility, or by some other detection means. When the smart packaging system 10 makes that determination indication reception at a healthcare facility, the smart packaging system 10 can run through internal checks to determine a status of itself and can take certain actions based upon its own status. When the smart packaging system 10 is received at the facility, the information transmitted can include storage location and expiration date.

As explained herein, the smart packaging system 10 can include one or more sensors that measure the environment in which the smart packaging system 10 is located, such as temperature sensors, moisture sensors, etc. Based on data collected by the sensors, the smart packaging system 10 can be aware of instances where the external environment resulted in potential harm to the smart packaging system 10. For example, if the smart packaging system 10 has a maximum temperature threshold of 100 degrees Fahrenheit, but the temperature sensor detects a temperature greater than that threshold, the smart packaging system 10 can assume that there is adverse damage to itself as a result of the temperature exceeding a safe threshold. Other sensors, as described, can be used to determine when the smart packaging system 10 has experienced some environmental condition that has likely resulted in harm to the smart packaging system 10, and the specific safety thresholds can vary depending upon the aspects of the smart packaging system 10.

If the smart packaging system 10 determines that it has experienced an unsafe environment, the smart packaging system 10 can respond in a number of ways. For example, the smart packaging system 10 can display a warning on a display 22 or via a transmission to a device in electronic communication with the smart packaging system 10. The warning could be a simple message, such as “DO NOT USE.” More specific details could then be provided upon request from another system, such as the HUB 50, a scanner, etc., in order to learn why the smart packaging system 10 has displayed such a warning. Those details could indicate that the package experienced, for example, too high of a temperature, which has now potentially jeopardized the smart packaging system 10.

In some variations, if an event is sensed by the smart packaging system 10, such as too high of a temperature, too much moisture, an expiration date is exceeded, the smart packaging system 10 experienced too great a force, etc., one or more operative capabilities of the smart packaging system 10 can be disabled. This disablement can occur at various stages. For example, if the smart packaging system 10 communicates with the HUB 50, such as for a status check or other reason, the HUB 50 can run a quick diagnostic test to determine whether the smart packaging system 10 has experienced any potentially harmful events. If a harmful event is detected, such as loss of sterility, a killswitch can activate within the smart packaging system 10.

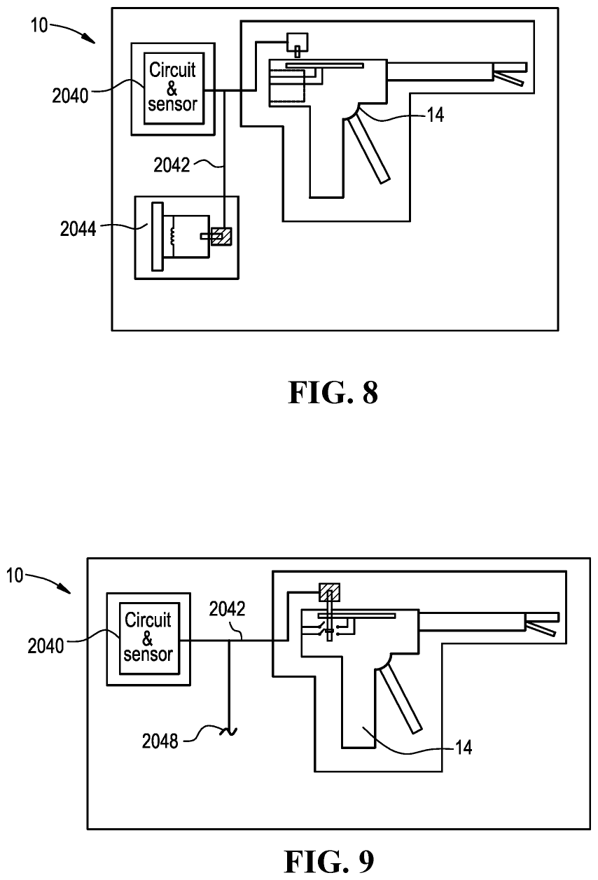

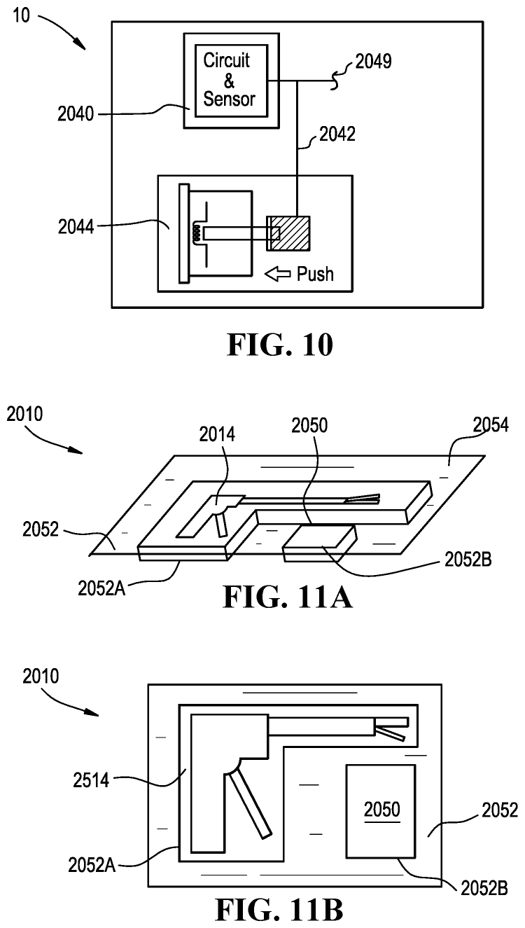

The killswitch can come in various forms, and examples can be seen in FIG. 8, FIG. 9, and FIG. 10. FIG. 8 depicts the smart packaging system 10, including a surgical instrument 14 electronically coupled to a sensor 2040 and circuit 2042 equipped with a battery 2044 and a solenoid 2046. Upon detection of a harmful event by the sensor 2040, a killswitch can be triggered in the smart packaging system 10. FIG. 9 depicts a first variation of the killswitch in which the solenoid 2046 is triggered to break a portion first 2048 of the circuit 2042 that transports power from the battery 2044 to the surgical instrument 14, thereby permanently disabling the smart packaging system 10. FIG. 10 depicts a second variation of the killswitch, except that instead of breaking the circuit 2042 to isolate the battery 2044, the solenoid 2046 breaks a second portion 2049 of the circuit to isolate the surgical instrument 14, thereby forcing the battery 2044 to drain at a rate that harms the integrity of the battery 2044. Other killswitches, not depicted, may include a piece of software that interrupts a boot sequence of the affected smart packaging system 10. Upon detection of a harmful event, the software prevents the smart packaging system 10 from booting properly, effectively preventing any use of the smart packaging system 10.

The smart packaging system 10 can be aware of when it enters healthcare facility storage as explained above. The smart packaging system 10 can include readable medium 20, of device information, and a label for the smart packaging system 10 can be generated by the healthcare facility that explains to the HUB 50 how to parse device information into a format that is compatible with the records system of the healthcare facility for automated reporting writing post-operational procedure involving the smart packaging system 10. In practice, as the surgical packaging system 10 is received at a healthcare facility, it can be scanned and immediately logged within the facility's inventory. Once in the inventory, the smart packaging system 10 can be monitored for events of interest, including recalls, expiration, first-in-first-out usage, etc. Monitoring can even include special use cases, such as when a procedure involving the smart packaging system 10 requires a high-stress application during the procedure. Selection of the smart packaging system 10 could occur based on the demands of the high-stress application, where an older smart packaging system 10 received by the hospital may not be prioritized for selection due to the potential for an increased operating risk associated with the performance of the high-stress application by the older smart packaging system 10. Instead, a more “fresh” smart packaging system 10 may be selected, despite the existence of a first-in-first-out usage policy.

The smart packaging system 10 can feature an adaptive expiration date, which can be programmed and stored on the smart packaging system 10. While a default expiration date can be provided, the adaptive expiration date can be adjusted based on environmental conditions measured by the smart packaging system 10. For example, if transit and/or a stockroom were very hot, the expiration data may come sooner than expected. The adaptive expiration date can adjust based on environmental data sensed by the sensors of the smart packaging system 10.

The smart packaging system 10 can indicate that it has expired in a number of ways, including a light indicator (e.g., green versus red), a non-powered indicator (e.g., a time-based label), scanning and being provided that information on a user device, periodic check-in by the HUB 50 with a corresponding notification to a user about expired products, the HUB 50 checking all smart packaging systems entering and exiting a stock room and informing a user of any expirations, and more.

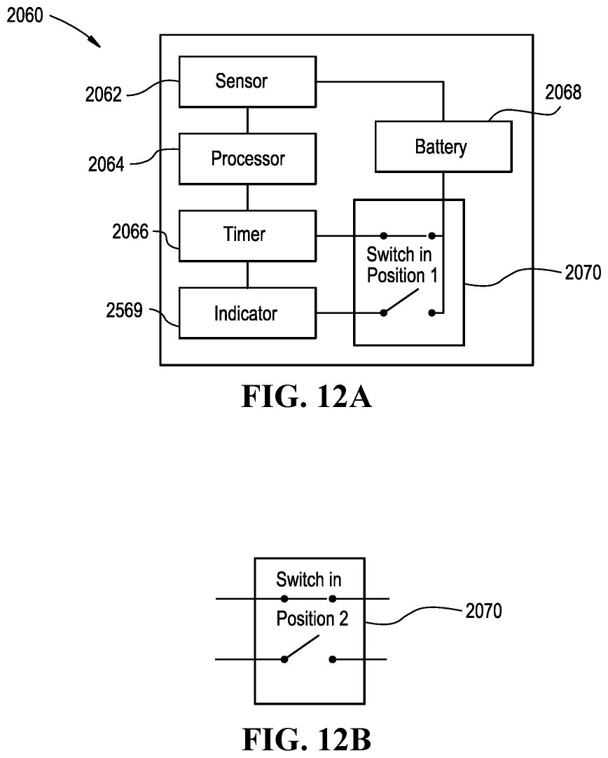

An example smart packaging system 2010 with a dynamic tracker 2050 can be seen in FIG. 11A and FIG. 11B. The tracker 2050 can be seen in greater detail in FIG. 12A and FIG. 12B. The smart packaging system 2010 can be identical to the smart packaging system 10 in any of the variations described herein. The smart packaging system 2010 can include a tray insert 2052 upon which contents of the smart packaging system 2010, including the surgical instrument 2014, in various compartments, with the surgical instrument 2014 resting in a first compartment 2052A and the tracker 2050 resting in a second compartment 2052B. A lid 2054 can be used to seal the tray insert 2052 to maintain sterility of the smart packaging system 2010. To check sterility of the smart packaging system 2010, a user can peel off the lid 2054 to expose the tracker 2050, while leaving the smart packaging system 2010 under seal.

The tracker 2050 can interact with a number of components to facilitate its operative capabilities. The tracker 2050 and components can be seen in the block diagram 2060 of FIG. 12A, which provides an illustrative and non-limiting example. The tracker 2050 components can include a sensor 2062, a processor 2064, a timer 2066, a battery 2068, and an indicator 2069. The sensor 2062 can be one or more of the sensors described herein, such as a temperature sensor, or other sensor capable of detecting environmental conditions that can affect the sterility and condition of the smart packaging system 2010. A switch 2070 can also be included, and when the switch 2070 is in a first position, as seen in FIG. 12A, a circuit between the sensor 2062, processor 2064, timer 2066, and battery 2068 can be completed. When the switch 2070 is in a second position, as seen in FIG. 12B, the indictor 2050 can also receive power from the battery 2068, and the tracker 2050 can show a time remaining until expiration and/or a current status of the smart packaging system 2010.

Although not shown, the block diagram 2060 can feature components to enable communication, wired or wireless, with other features of the smart packaging system 2010, HUB 50, and any other type of device described herein.

The smart packaging system 10 can be aware of when it is requested for usage in a medical procedure. The smart packaging system 10 can be aware of the kinds of procedures for which it is typically used, the types of equipment used in those procedures, its operational capabilities and requirements, and more. As a result, the smart packaging system 10 can recommend, via the display 22, an electronic notification, etc., of a preferred storage location within a healthcare facility, which could include environmental factors, proximity to other compatible products, etc.

The smart packaging system 10 can include labeling to enable the smart packaging system 10 to customize integration of the smart packaging system 10 into the healthcare facility's operations. The labeling could include a scannable code of device information and a second label generated specifically by a hospital that tells the HUB 50 how to parse device information into a format used by that facility to feed into records for report writing post-procedure.

If a smart packaging system 10 becomes aware that it has been requested for a specific procedure, the smart packaging system 10 can also know that, typically, certain equipment is used in conjunction with the smart packaging system 10. Some of these other equipment can be smart equipment that are capable of communicating with the smart packaging system 10, the HUB 50, and other devices. When the smart packaging system 10 detects incompatibility, it can provide an alarm or notification of that incompatibility. However, in some instances, a doctor may pull a seemingly peculiar combination of devices. The smart packaging system 10 can ask the doctor if they would like to register the combination as a new bundle for future reference.

When the product is pulled for use, the information transmitted by the smart packaging system 10 can include cart number, item number, destination, whether another stock room visit is needed, doctor name, etc.

The smart packaging system 10 can be aware of when it enters an operating room. If the smart packaging system 10 is confirmed for usage in a procedure, no alarms, warnings, or other contraindications will be issued. The smart packaging system 10 can display a message indicating readiness for the procedure, such as a specific word like “READY” or a symbol such as a green checkmark meant to indicate readiness. The various smart packaging systems 10 used in the procedure can also alter their displays 22 to reflect a usage order (e.g., 1/5, 2/5, etc.) that is typical for the operating procedure.

If the smart packaging system 10 enters and operating room and an alarm or warning is issued, or some other message, such as an indication of a preferable alternative, the display 22 or display 22 of the smart packaging system 10 can provide an indicator such as “DO NOT OPEN UNTIL ALL CLEAR,” or other message, symbol, etc. Such a message could also be displayed on a smart packaging system 10 that should not be opened until a specific time in the procedure, even if the smart packaging system 10 is otherwise all clear.

For a given procedure, the HUB 50 or other system can display an indication of preparedness. The indication can reflect a proportion of ready components needed for the surgical procedure, and the indicator could appear as a loading bar, for example. There can also be an indicator of the overall procedure, such as a timeline indicating when specific smart packaging systems 10 should be used relative to each other. If certain information is critical to the procedure, that information can be displayed in an obvious manner, such as in an apparent location, at an appropriate size, and with additional indicators such as flashing lights, an audible alarm, etc. Certain information indicators can also be transitory, such that if they are ignored for a certain amount of time, they disappear.

The smart packaging system 10, as part of its ability to receive and transmit electronic information, can be systematically tracked when the smart packaging system 10 is located in a healthcare facility. The smart packaging system can be tracked using a management system for “floating” stock management that is virtual and non-continuous, but that allows for the instant retrieval of the location, identification, and tracking of all similarly-situated smart packaging systems 10 within a specific healthcare facility. This kind of management system can take advantage of the capabilities of the smart packaging systems 10 as described herein in order for the HUB 50 or other similar management tool to plan procedures and circulate stock as needed based on the HUB's 50 and smart packaging system's 10 situational awareness of the procedures, staff, and schedule of the healthcare facility. This can allow for the healthcare facility to stock less smart packaging systems 10 overall because specific smart packaging systems 10 with their unique contents can be tracked and located at a moment's notice without the need to keep excess product on-site.

This management system and various applications thereof, including as related to capabilities of the smart packaging system 10, will be described below.

The smart packaging system 10 can possess dynamic awareness of its location and distribution throughout its lifecycle. For a given smart packaging system 10, the surgical instrument 14 and contents of the smart packaging system 10 can be tracked within a healthcare facility as a result of the communicative capabilities of the smart packaging system 10 and of the surgical instrument 14, as well as the HUB 50. This can allow the smart packaging system 10 to be located not only within a specific stockroom within the healthcare facility, but also in a hall, on a cart, on a return table, is a misplaced location, etc.

As a result, the management of the smart packaging systems 10 does not necessarily need to rely on a physical stock room, but instead, the management system can rely on a virtual stockroom, which can take various forms, such as an electronic database of various smart packaging systems 10, their location, their operational capabilities, etc. This virtual stockroom could include preset locations, such as physical stock rooms located within the healthcare facility, and smart packages could be stored in these dedicated stockrooms generally. However, the virtual stockroom can act as an overarching management tool that can be updated in real time to reflect current information of the various smart packaging systems 10 in the healthcare facility.

In an example, if two smart packaging systems 10 are sent to an operating room for selection by a surgeon between the smart packaging systems 10, the unneeded smart packaging system 10 can be placed on a cart to be returned to a stockroom. If that smart packaging system 10 is needed in another operating room before the cart can return the smart packaging system 10 to the stockroom, tracking down that smart packaging system 10 can be difficult in a traditionally-managed healthcare facility. With the virtual stockroom, the smart packaging system 10 can be constantly tracked and the cart can be diverted to the second operating room so that the smart packaging system 10 can be put to use immediately without the need to restock the smart packaging system 10 in a stockroom first. This can greatly improve efficiency within a healthcare facility.

With this kind of management system, unopened smart packaging systems 10 can be diverted to a different operating room or patient based on any kind of need, priority, timing, etc. Further, suitable alternative options can be quickly located and provided when a first choice for a smart packaging system 10 is not available. Such alternative operations could easily be located within the healthcare facility thanks to their presence on the virtual stockroom database.

In another aspect, this kind of management system can provide for automated stock location and circulation. For example, delivery of stock could become automated, such as by a robot device. In some healthcare facilities with automated stock management robots and other kinds of robots, this type of management system can allow for the location of a specific smart packaging system 10 to be provided to such a robot and the robot can then go and retrieve the smart packaging system 10 wherever it is located.

The need for physical stockrooms located in fixed locations could be reduced or eliminated altogether with the addition of specialized robotics systems, virtual stock management systems, and/or similar systems. These new stock rooms could be a series of mobile “cabinets” with a series of smart packaging systems 10. As a smart packaging system 10 is needed, the “cabinet” (essentially a robot with a lot of storage) could travel to the location where the smart packaging system 10 is needed, and the smart packaging system 10 could be delivered automatically. Such automated delivery could be made to stop for various scenarios, such as within a certain amount of time prior to a procedure, upon detection of the opening of a smart packaging system 10 such that an alternative or automated delivery is no longer needed, etc.

This system could further control management of smart packaging systems 10 based on a number of factors and prioritize movement thereof accordingly, such as by keeping commonly-used smart packaging systems 10 in more convenient locations to allow for quick delivery, as well as by supplying smart packaging systems 10 on a first-in first-out basis to reduce the likelihood of expired smart packaging systems 10. Alternatively, specific expiration dates of smart packaging systems 10 could be tracked so that smart packaging systems 10 are provided with expiration in mind.

In some variations, the management system could employ the use of one or more “smart” operating rooms, which can be used to catalogue devices, supplies, and products that enter into them. If, for a given procedure, the catalogued items are not consumed, they can be gathered together to leave the operating room to be returned to storage. As the items leave, the management system via the HUB 50, for example, can add the items back to the virtual stock database for future use.

As smart packaging systems 10 are moved around a healthcare facility during management thereof, the smart packaging system 10 can display, via the smart display 22, display 22, etc., information related to that management. For example, the smart packaging system 10 can display an operating room number where it is scheduled to be used.

To make the management of smart packaging systems 10 more efficient, the management system can reroute smart packaging systems 10 so that they are not moved in an inefficient manner. For example, if a smart packaging system 10 is originally requested for use in a first location but then it is needed in a second location, rather than sending the smart packaging system 10 back to the stockroom first before sending it to the second location, the smart packaging system 10 can be routed directly to the second location.

The management system can employ the use of smart carts that can be included in the virtual stockroom. The smart carts can have the ability to determine the product or products on them and also to provide where they, and their products, are located. The smart carts can have a unique digital ID as part of their inclusion in the virtual stockroom, and they can be tracked within the management system. The smart carts can be equipped with their own display means, such as the kind found on the smart packaging system 10, and the smart carts can display information related to the products they carry, the cart destination, plan, etc. The smart carts can also be equipped to identify personnel in proximity to them, such as by detecting a smart badge or other means. The smart cart can inform identified personnel of certain information, such as to relay messages, make requests, and more. For example, the smart cart can instruct personnel to remove certain products from the smart cart to complete a delivery.

In some variations, the smart carts can be maneuverable by a person or the smart carts can be drivable or self-driving when moving products to various locations within a healthcare facility. The HUB 50 can track the smart carts as they pass by beacons or as they communicate electronically with other in-network equipment. With this tracking, the smart carts can directed to various locations, and personnel at the healthcare facility can be directed to the carts as needed in order to move products around the healthcare facility. In some aspects, a smart cart can be free-floating, and can be moved as needed to key areas of the healthcare facility.

Further, the smart carts can be equipped to display instructions for their next destination, such as a specific operating room in the healthcare facility. The instructions can also include a list of products to be delivered to the set location upon delivery.

In some aspects, the smart carts can be specialty-dependent, i.e., all products on a given smart cart are related to cardiovascular operations. The smart carts can be colored or otherwise marked to indicate this specialty in order to inform observers of the general contents of the cart.

The management system can aggregate smart packaging systems 10, and the smart packaging systems 10 can coordinate between themselves in order to determine priority, status, and location, among other information. This communication between smart packaging systems 10 can occur any time that two or more smart packaging systems 10 are within range for electronic communication to occur, and in particular, it can occur when the smart packaging systems 10 are located on the same smart cart, or in the same physical stock room, operating room, etc.

If a smart packaging system 10 is unexpectedly taken off of a smart cart, a notification can be transmitted to the HUB 50 by any capable system aware of the removal from the smart cart to indicate to the HUB 50 that the specific smart packaging system 10 that was removed cannot be used because it is on the move. A replacement product or component can be requested to be delivered to that specific smart cart from which the smart packaging system 10 was removed in order to minimize disruption to planned procedures.

The management system can employ a priority system or hierarchy among devices of any kind. Devices of more importance can be tracked more closely than devices of less importance, and if there is ever an issue with the management or delivery of two devices, preference can be given to the device deemed more important by the priority system. For a given smart cart, as packages are removed and delivered, eventually a certain threshold is reached or a window in a delivery schedule is available in which the smart cart can be restocked. With the current management system, restocking can be coordinated to be even more efficient. For example, given two smart carts beginning their day with nearly identical products aboard, and given that one cart becomes short on a certain kind of product and needs to restock, while the other cart has an excess of that product, the two smart carts can coordinate their restocking with each other, such that the cart with excess product can resupply the cart that is low on that product. This can eliminate an extra trip to a stockroom.

Expiration dates of smart packaging systems 10 can be tracked, and when an expiration date nears, that smart packaging system 10 can be given increased priority to minimize waste.

Where backup stock or an alternative product is needed, the smart carts can direct a user to the alternative product. This direction can come in the form of pointing a user to another cart with the alternative product, pointing the user to a specific stockroom containing that product, etc.

While certain items may not traditionally possess “smart” capabilities, all packaging can be equipped with an RFID tag or equivalent device to provide a minimum threshold of “smart” functionality, which can enable at least a basic level of management.

In some variations, the smart carts can display an active inventory of the items currently located on them. This information can be found on a display or other user interface, which can also be navigable, such as by touchscreen, buttons, or other input, in order to provide a user with information in an organized and meaningful way.

Although planning and scheduling can be implemented to guide smart carts as they are needed, based on various information related to surgical procedures, stocking, etc., scheduling can change quickly. When scheduling changes and needs change as a result, the management system can adjust to reroute carts to where they are needed based on the updated information. This information can be provided to a smart cart in the form of an update or an alert to communicate to a handler the updated pathway, or, if the smart cart is autonomous, the cart can receive the updated route information and then automatically respond. This type of response can reduce confusion associated with schedule changes and the related product coordination for the updated procedures.

In some variations, the smart carts can be modular and can be aware of common aspects of products in order to optimize handling and delivery. The smart carts can come equipped with attachable and re-attachable sub-sections in the form of trays, drawers, etc., which can be packed with smart packaging systems 10 or sets of products in specific arrangements, such as for a given procedure, etc. The sub-sections can be attachable in a number of ways, such as with a series of magnets, by latching, or by other means. This can allow for the quick swapping and stocking of smart carts with specific kits or related groupings of products. The sub-sections could be grouped by functionality as well, such as having a suture sub-section, a heart sub-section, etc. The sub-sections themselves could be equipped with smart capabilities, and can be capable of identifying compatibility in products stored within them. In some variations, each of the sub-sections can be packaged with all of the smart packaging systems 10 and accessories needed for a given procedure. When the smart cart arrives at the corresponding operating room, the entire sub-section can be removed and taken into the operating room.

In some variations, smart carts can help mitigate incompatibility issues between smart packaging systems 10 and other components by offering a secondary check. If an attempt is made to take an incorrect device off a smart cart, the smart cart can recognize the taker, such as via their smart badge, and inform them that the product they grabbed is incorrect. For example, if the person grabs a certain smart packaging system 10 needed for a procedure by they also grab an accessory used with a different smart packaging system 10, the smart cart can inform them that they need to actually grab a different accessory on the cart.

To make these personnel-based determinations, the smart cart needs to know and understand who is associated with a given operating room at a given time. For a procedure occurring in a given operating room, the identities of personnel, such as surgeons, nurses, etc., associated with that specific procedure can be generally known for staffing and coordination purposes. If a smart cart is making a round of deliveries and one delivery is to that given operating room, the smart cart can read a smart badge of a personnel accepting a delivery, associate this person with a specific delivery of one or more smart packaging systems 10, and then instruct the person to take the corresponding one or more smart packaging systems 10 off the cart.

Further, a smart cart can also recognize a “clearance level” of personnel in order to determine who can receive or claim a certain smart packaging system 10 off of the smart cart. For example, if the smart cart is handling package containing sensitive information, a controlled substance, etc., the smart cart can check a clearance level of a person claiming that package and react appropriately. In the case of an improper retrieval by a person without clearance, this reaction can include notifying another person, such as a person with authority to rectify the situation, preventing the person from claiming the device (e.g., through the use of smart locks on sub-sections of the smart cart), disabling or locking the smart packaging system 10 directly, etc. Further, the smart cart can also simply record the identity of the person who took the smart packaging system 10 and that smart packaging system 10 can be further tracked. That information can be provided as needed.

In some variations, the smart carts can be programmed with a scheduling awareness and can be optimized based on a layout of a healthcare facility, including key locations such as stockrooms, operating rooms, etc. More specifically, the smart carts can be programmed with a daily schedule, which can take into account planned surgical procedures, procedure times, other smart carts, and other information. Based on this programmed information, the smart carts can determine routing, path optimization, restock windows, and more. For example, if a smart cart knows that it will cross paths with another smart cart stocked with a certain product at a certain time, and it also knows that it needs that certain product at a later time, the smart cart can grab the product from the other smart card when the two smart carts cross paths. This type of routing based on not only the location of store rooms but also the location and routes of other smart carts can greatly increase efficiency in stock management at a healthcare facility.

The smart carts can be sub-divided into at least two categories, which can include stationary floating carts and mobile floating carts. Mobile floating carts can have a set pathway based on the time of day, location for delivery, etc., and routing of the mobile floating carts can be updated based on procedure times. Stationary floating carts can be placed or moved to key locations and can serve as a pick-up point for various smart packaging systems 10 and accessories.

Depending upon timing, scheduling, and other variables, such as current stock located on a smart cart, a smart cart can shuffle its stock into a certain order. The smart cart can be equipped with a system to move its components, such as with a drive mechanism that operates a vending machine-like contraption, or another contraption that operates like a conveyor belt.

As part of smart packaging system 10 management, the virtual stock room can create digital kits within the virtual stockroom or supply chain on the whole. These digital kits can tag each smart packaging system 10 with a digital indicator marking it as part of a package or bundle of smart packaging systems 10 associated with a given procedure. For example, if a routine procedure requires five specific kinds of smart packaging systems 10, those kind of smart packaging systems 10 can be marked with an identifier linking them to the routine procedure as part of a kit for that routine procedure. Smart packaging systems 10 that are of the kind used for that routine procedure can be permanently associated with that kind of kit for that routine procedure, and it the smart packaging system 10.

To assist in management, smart packaging systems 10, including surgical instruments 14, accessories, etc. can be identified and tagged as being commonly used together, such as for a given procedure or as a result of some functionality of one of the surgical instruments 14. With this knowledge, adjustments can be made organizationally to co-locate commonly-pulled smart packaging systems 10 in storage to minimize efforts during retrieval of the commonly-pulled smart packaging systems 10. For example, if a surgical instrument 14 is always used in conjunction with a specific type of suture, those two items can be organized together such that, when they are needed for a procedure, the items are physically located near one another to minimize effort in tracking them down.

To further assist in tracking down smart packaging systems 10 in a certain kit, the smart packaging systems 10 can give off an indicator. The indicator can be an auditory indicator and/or a visual indicator. Audible indicators can be in the form of a tone when certain devices in a kit are placed together in proximity, thereby indicating that they have been properly sourced. A different tone can be used to indicate a mismatch or incorrect pairing. The respective tones can be happy or sad to further indicate the purpose of the tone. Audible tones can be used to assist in general location of a certain smart packaging system 10. Visual indicators can be in the form of a color-coded label or other special marker to indicate that the smart packaging system belongs to a certain kit, or in the form of a flashing light to assist in location of the smart packaging system 10. This type of indicator can be based on user preference and can be adjusted as needed.

In certain variations, augmented reality-enabled smart packaging systems 10 can be used. In these variations, locations can be indicated via the augmented reality (AR) and displayed on a tablet, a wearable such as glasses or a watch, a phone, or other system. Certain combinations may show up as different colors in the AR environment, and indicators showing compatibility and/or incompatibility with other systems can be provided in the AR environment. The AR environment can cross-check with other systems described herein to confirm any information necessary, as well as to supplement when any given system fails to fulfill a request.

When smart packaging systems 10 are grouped together into kits, additional features can be employed. For example, coupled products can automatically be ordered in batches according to their kit. The kits also provide the HUB 50 with a convenient list of items, which can be used as a checklist in other contexts. The kit can also describe alternatives to any of its members, which can be quickly referenced if a specific item in a kit is unavailable. Further, when smart packaging systems 10 are constantly pulled with other smart packaging systems 10, the smart packaging systems 10 can begin to recognize patterns that develop, including their association with other smart packaging systems 10. This can be done in a variety of ways, such as machine learning or pattern recognition programs, among others. When certain patterns are recognized, the smart packaging system 10 can make suggestions to update or create virtual kits based upon the recognized patterns.

Additionally, users, such as healthcare personnel, can create their own customized kits, which can be based on a variety of factors, including preference, availability, cost, etc. Based on these customizations, as well as other recommendations, kits can be offered by a manufacturer to a healthcare facility so that the facility can purchase kits in bulk and/or at a discounted rate, while ensuring that the members of the kit are compatible. Other benefits can be provided as well.

One such benefit includes the ability to pair smart packaging systems 10 belonging to a kit, which can provide the ability to transfer data to optimize an algorithm used in the operation of one or more of the smart packaging systems 10. For example, if a harmonic blade is used first in a procedure, it can identify a tissue type/integrity and relay information to a stapler (which is part of the same kit) as to what staple delivery method is best suited to the tissue type. The harmonic blade could also suggest which stapler cartridge to use. Where the stapler is used first in a procedure, the stapler could provide certain information to the harmonic blade. Additionally, an adjustment of functionality could be transmitted between the devices based on information gleaned from one device. This information could be communicated and coordinated via the HUB 50, and it could be displayed to a user or other personnel, such as via a display screen in the operating room. Unless devices are properly labeled as belonging to a kit, this functionality could be disabled in order to encourage the use of such virtual kits. In some variations, devices belonging to a kit, or which can be made to join a kit, can prompt a pairing process upon detection of a nearby device belonging to the same kit.

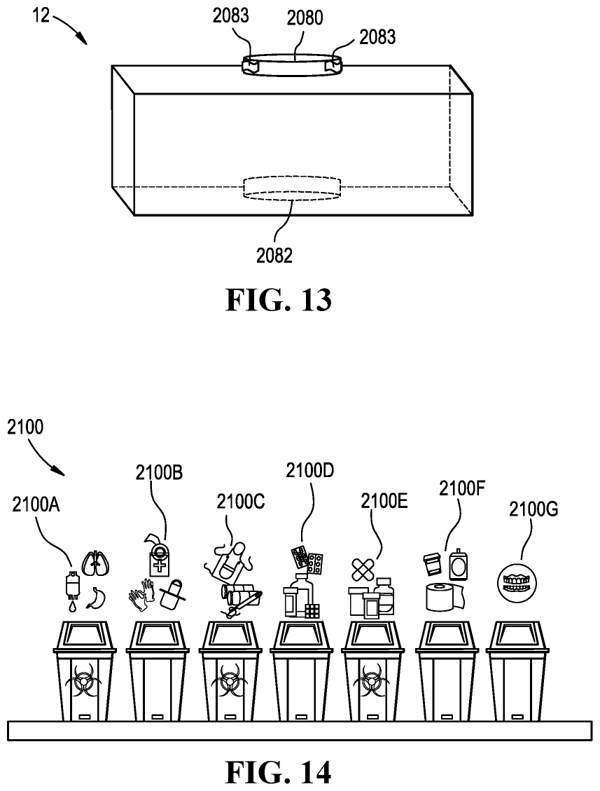

The packaging 12 of the smart packaging systems 10 can include features and design elements can make the various smart packaging systems 10 more physically compatible. Packaging 12 that can be linked together in bundles could reduce the chance of selecting an incorrect or incompatible device, or running out of a certain device in stock. For example, the packaging 12 could be designed such that it nests into other packaging and takes up less space as a result, which can be seen in FIG. 13, for example. The packaging 12 is shown including an extension 2080 and corresponding depression 2082 (shown in phantom) that can lock packaging 12, such as via a snap fit, for example. This design allows the packaging 12 to be stacked together. Further, the points of contact could be electrical contact points 2083 that link together the smart packaging systems 10 electronically to enable communication between the joined smart packaging systems 10. While only one extension 2080 and one depression 2082 are shown, multiple extensions 2080 and depressions 2082 could be included, and at various positioned on the packaging 12, including top/bottom and/or sides. Further, the extensions 2080 and depressions 2082 could have various sizes, shapes, and patterns, and more, including regular, irregular, and/or fanciful shapes such as logos.

The smart packaging systems 10 could further link to a smart pallet or other smart carrier device, such as the smart cart described above. The pallet or equivalent could be reusable and include onboard power and memory, as well as means to connect to the smart packaging systems 10, such as via the electrical points of contact described above, wireless communications described herein, or any other known method. The smart pallets could be further capable of communicating with the HUB 50 and receiving data relevant to the smart packaging systems originally transferred to a healthcare facility thereon. The smart pallet could then be returned and the data could be collected as needed. As described herein, disposal procedures for smart packaging systems can be complex and varied, and such procedures could integrate a smart pallet for reclamation processes.

In variations where the smart packaging systems 10 are linkable, the unique identifying features of each individual smart packaging system 10 can be placed such that they are individually legible even when packaged and linked with each other.

In some embodiments, the smart packaging system 10 has an awareness of waste management procedures in order to provide a user with approved disposal and reclamation instructions related to contents of the smart packaging system 10. The smart packaging system 10 can include packaging aspects that provide interactive waste stream guidance that enable the storage, reuse, and breakdown of contents of the smart packaging system 10 for disposal, and the packaging aspects can grant the smart packaging system 10 the capabilities to determine the proper waste stream guidance.

The smart packaging system 10 can provide disposal methods, disposal recommendations, and general disposal management information to a user. These can include instructions or steps for disassembly and disposal; documentation of usage, product operability, and aspects related device utilization; and tracking disposal processes.

The smart packaging system 10 can provide instructions for disassembly and disposal of the components of the smart packaging system 10, which can vary based on a number of factors. For example, the instructions could follow the standard guidelines for waste management, with healthcare waste segregation involving the separation of various wastes into colored bins according to a waste classification. These standard guidelines can be seen in FIG. 14, for example, and they include colored bins 2100 marked with: red 2100A for anatomical waste (e.g., blood and organs; orange 2100B for clinical/infectious waste; yellow 2100C for clinical/highly-infectious waste; blue 2100D for medicines (e.g., unused drugs); purple 2100E for cytotoxic and/or cytostatic products (e.g., chemotherapy medicines); black 2100F for municipal waste (i.e., not clinical or medical waste); and white 2100G for dental waste. Healthcare waste segregation is ideally performed at the first opportunity possible to prevent hazardous waste from mixing with regular waste. Providing explicit procedures and instructions for the proper disposal and management procedures can decrease potential risks of contamination.

The standard guidelines could be presented to a user in a number of ways. In some variations, every label of every packaging of the smart packaging system 10 could having some designator indicating the proper method of disposal. For example, if a surgical instrument 14 contains cobalt, a regulated substance with procedures varying based on local regulatory schemes, a label for that surgical instrument 14 could indicate the proper waste management guidelines according to the relevant local regulatory scheme as it pertains to cobalt. The label could also provide instructions for a user to act according the proper guidelines. In some variations, the packaging could feature color coding according to universal disposal guidance, such as the kind described above with respect to FIG. 14. In some variations, an LED light, or equivalent, on the smart packaging system 10 or any of its components could indicate in which bin to deposit that component (e.g., a red LED means place the component in the red bin 2100A). The LED light could change based on whether the packaging was opened or the device was used. If the device was used and became contaminated, the LED light could indicate the relevant bin. If unused, the LED light could indicate a different color corresponding to a bin for components that went unused during a procedure. To incentivize proper disposal of components, disposal guidance could be worked into a contract with a healthcare provider, (i.e., if a certain percentage of waste is reclaimed, a corresponding discount will be provided). Guidance of reclamation procedures can be provided to healthcare provider to assist with compliance.

In some embodiments, the smart packaging system 10, the surgical instrument 14, and/or the packaging can read product info of surrounding devices contained in a disposal bin. This could be done in a number of ways, including with RFID tag detection of other tags in close proximity. For example, if a certain device or component is disconnected from the HUB 50 (indicating disposal) and that device or component then detects that it is near components which should belong in a separate disposal location, this could indicate that one or more of the device or components were disposed of incorrectly. If a certain component is detected and other components that belong in a separate container are not detected, this could indicate that proper disposal procedures were followed. In another example, if, after disposal, a battery or battery-carrying component is detected nearby, improper disposal could have occurred. If such a scenario of improper disposal is detected, the detecting component could transmit that information to a user or to the HUB 50, following a reconnection, and inform the user of the error. If repeated errors are detected, or a pattern emerges indicating improper procedure, a supplier could be informed through any appropriate communication channel.

If the disposal options of a healthcare provider are limited, the smart packaging system 10 can adjust the instructions provided based on the available options. This can be done in a number of ways, and may involve, for example, setting a hierarchy among the disposal options, such that if one option is unavailable, the smart packaging system 10 will select the next option, proceeding down the list until an option is available. If no option is available, the smart packaging system 10 can inform a user to follow their standard procedures for that unavailable aspect.

During disassembly of contents of a smart packaging system 10, complexity of disassembly can be an issue. Accordingly, it can be beneficial to simplify procedures in order to reduce the likelihood of error, which could result in harm. In some variations, the smart packaging system 10 can include a numbering scheme for disassembly to provide an order in which disassembly should take place (e.g., first remove a battery, second isolate a blade, etc.). The smart packaging system 10 could include one or more indicators, such as an LED, that activate after each step in the scheme is complete, in order to provide a visual indication of progress. In some variations, dynamic labels, such as the smart display 22 (e.g., e-ink labels) or the display 22, could provide step-by-step instructions for disassembly. When a disassembled component is properly disposed, the smart packaging system 10 could update the display 22 or notify a user to activate the next step. The smart packaging system 10 and the HUB 50 can be in communication with each other, as explained herein, to monitor progress and update the displayed instructions for disassembly. In some variations, the smart packaging system 10 can include specific surgical instrument 14 instruction data for a specific geopolitical location. For example, based upon a current geopolitical location of the smart packaging system 10, the instructions may vary to accommodate relevant local regulations. In some variations, a compliance assurance service can be used to track device disassembly and disposal to confirm that specific components, like batteries, are properly disposed.

When instructions vary depending upon a geopolitical location and/or specific healthcare provider regulations, a determination of the location, including of the specific healthcare provider, could be made based on a product code or some other identification (i.e., it is known that this product code is being shipped to a specific location so the instructions will correspond to that location). The determination could also be made through communication with the HUB 50.

Alternate disposal methods and options can also be provided. As explained above, a hierarchy of options can be used such that if one option is unavailable, the next option in the hierarchy is selected. If an error is detected during use of a surgical instrument 14, for example, an alternative disposal procedure can be implemented. This procedure can proceed by asking whether there is a battery included in the surgical instrument 14, and, if so, receiving an indication, either by a system or a user, of whether the battery was at issue. If the battery is at issue, the surgical instrument 14 can be reclaimed without the battery, and instructions can be provided to effect this reclamation. If the battery is not at issue, the surgical instrument 14 can be reclaimed with the battery, and instructions can be provided to effect this reclamation. The query can proceed by moving on to the next possible component, and reclamation procedures can be adjusted based on user indication of a faulty component. In some variations, a message can be transmitted and/or displayed by the smart packaging system 10 and/or HUB 50 to inform a user to return the smart packaging system 10 according to packaging instructions. Upon the detection of an error, data surrounding the error can be recorded and reported to a supplier or relevant party in order for the error to be assessed, such as for correction purposes.

If a component of the smart packaging system 10 is determined to be unusable, either by detection by the smart packaging system 10 itself, the HUB 50, or by indication of a user, the alternative procedures can be provided. Knowing when the component was deemed to be unusable can also assist in assessing fault. For example, if the error occurred during transit, a healthcare provider would not be at fault. In some variations, alternative procedures can be triggered based upon the context of use. For example, if the problematic use occurs during a clinical trial, the reclamation procedures may vary in order to reclaim more components to receive as much information from the clinical trial as possible, including information related to operational errors.

As explained above, the smart packaging system 10 can document usage, operability, and aspects of device utilization. In some variations, the HUB 50 can write permanent information to the smart packaging system 10 related to the above documented information.

As explained above, the smart packaging system 10 can assist in tracking disposal processes. As also explained above, the smart packaging system 10 can store a list of all of its contents, including components and sub-components of the contents. The smart packaging system 10 can create a list of all products that entered the operating room. When a disposal process begins, the smart packaging system 10 can create a certificate of destruction (COD) for all supplies consumed during the procedure. After a procedure occurs, the HUB 50 can record and cross-check the anticipated disposal instructions for each product against the products actually being disposed. If a deviation is detected, a notification can be transmitted to a user. If the HUB 50 recognizes that a package was unused during a procedure but it was placed into a disposal area, the packaging can signal to the HUB that it was unopened and healthcare personnel can be notified to return the unused package to a stock room. If the HUB 50 determines that a battery was not properly disposed of, or example, or the battery was improperly packaged for return, the smart packaging system 10 and/or the HUB 50 can warn a user and require acknowledgement by the user before proceeding with additional operations.

In some variations, the HUB 50 or smart packaging system 10 can provide labels or digital metadata related to disposal packaging that provides instruction for destination, method of destruction, special handling instructions, and other related information, to a user. This information could be provided in a number of ways, such as via the dynamic display 22, which can be made removable and re-attachable to return packaging. An RFID tag 16 or similar device can detect the packaging change and update the label to display the information.

In some embodiments, the smart packaging system 10 can track waste streams and disposal progress in order to determine compliance with disposal procedures. To assist in determining compliance, the smart packaging system 10 can determine if the components to be disposed qualify as regulated medical waste, per the relevant local regulatory scheme, or larger regulatory scheme (e.g., the Medical Waste Tracking Act (MWTA), and the Resource Conservation and Recover Act (RCRA) in the United States). The smart packaging system 10 can further determine site-specific regulations, including those relevant to the current geopolitical location of the smart packaging system 10. For example, in the United States, knowing that the smart packaging system 10 is in a United States facility may not be enough, as regulations could vary state to state. Knowing the specific state may be necessary to determine appropriate disposal procedures. Relevant regulated medical waste can include, pathological waste, human blood/blood products, microbiological waste, contaminated sharps, isolation waste (such as from highly communicable diseases), animal waste, etc.

In some variations, the smart packaging system 10 can determine if the components to be disposed qualify as batteries, electronics, heavy metals, or other similar materials. If a determination is made and confirmation is received that the components do indeed qualify, procedures can be provided, including to return the device to a supplier for proper disposal, if no other options are available.

In some variations, the HUB 50 can record smart packaging system 10 disposal data. This data may be used to satisfy contract stipulations with the healthcare provider centered on disposal procedures, which may result in penalties or incentives for the healthcare provider.

In some variations, the HUB 50 can flag specific components of the smart packaging system 10 and inform a user when a proper disposal bin is not available.

In some variations, the smart packaging system 10 can provide the HUB 50 information on any components or material within the smart packaging system 10 that has been flagged, enabling the HUB 50 to return any relevant disposal requirements of the facility, region or waste management company. The smart packaging system 10 can update the listed disposal instructions to reflect current information and/or best practices. For example, certain specialty waste management instructions may be needed for electronic circuit boards, batteries, local adjustments, location of special handing of REACH materials, and device/packaging disposal generally.

In some variations, the smart packaging system 10 can track recycling value and provide that information to a user. For example, the metric could be: if certain components are used, they must be recycled properly. To incentivize proper disposal, the smart packaging system 10 could display or provide a measure of the financial savings of the recycling, an indication of progress toward a greater recycling goal (e.g., 15% of recycling goal for Q1), and the HUB 50 could also inform the contribution that recycling a certain component would have on the greater recycling goal. The smart packaging system 10 could also employ a reclamation credit system. For example, a tracking or progress of the reclamation could be displayed or provided to a user, and certain rewards could be tied to reclamation (e.g., reclaim 10 devices and get a certain amount of credit). The rewards could be exponential to incentivize reclamation. The credit could be awarded upon receipt of the reclaimed device. In some aspects, the display 22 can be removable such that the label stays with the reclaimed device such that the data recorded by the label is also reclaimed.

The smart packaging system 10 can include features to assist with reusability, disassembly, and/or return of contents of the smart packaging system 10 following use.

These features can include aspects of the smart packaging system 10 that facilitate the temporary storage of components and contents of the smart packaging system 10 and that facilitate the re-packaging of components and contents of the smart packaging system 10. In some instances, it is desirable to reclaim components of the smart packaging system 10, such as for assessment of performance, error processing, recalls, etc. To assist in reclamation of components, the smart packaging system 10 can include return packaging. This return packaging can be reusable packaging that is re-tasked for disposal or return of the contents of the smart packaging system 10 after use, as well as being the system by which a product can enter a sterile field during a surgical procedure involving the smart packaging system 10.

For example, the packaging 12 can open up to a form such that it can rest on the top of a disposal bin. Once the surgical instrument 14 has been fully used and is ready for disposal, the surgical instrument 14 can be resealed into the packaging 12 and immediately fall into the waste bin. In some variations, the packaging 12 can be positionable in more than one orientation, such as on a bottom and on one or more sides, while still providing access to its contents. In this way, the packaging 12 can take up a smaller footprint. To assist with the re-packaging process, the packaging 12 could come with a resealing means, such as adhesive strips located on selective flaps of the packaging 12 that are covered by a seal. When re-packaging is desired, the seal can be removed to expose the adhesive and allow for the flap to be closed by adhering the flap to the packaging 12 via the exposed adhesive.

To assist in disposal, the smart packaging system 10 can include features that interface with disposal containers, which can be located in an operating room hosting a procedure involving the smart packaging system 10, or they can be located on-site at a healthcare facility. Items placed into the disposal containers can be disposed of as is proper for their contents. In some variations, the disposal containers can come in various forms, including as re-sealable packaging used for contamination control in disposal. In some variations, the disposal containers can be a waste stream container that is designed specifically for the kind of waste stream through which it will be processed, as described above with respect to FIG. 14, for example. For example, if the waste stream container is meant to be used in the disposal of organic waste, the waste stream container can be made of a non-reactive, non-porous material, such as various plastics, in order to properly transition the organic waste to its ultimate disposal location (e.g., an incinerator). The disposal containers can be provided by the original equipment manufacturer (OEM), depending upon the contents of the smart packaging system 10. However, the disposal containers can also be used to store non-OEM waste where appropriate. If non-OEM equipment is used in a procedure with OEM equipment, the instructions and procedures for disposal and/or reclamation, as explained herein, can indicate to a user that the non-OEM equipment may have unknown waste management requirements.

In some variations, the disposal containers can feature lids that provide temporary storage for the packaging 12. When a procedure is complete, the lid can be quickly used to dispose of the stored contents. In some variations, the packaging 12 can magnetically attach to a side of the disposal container for temporary storage.

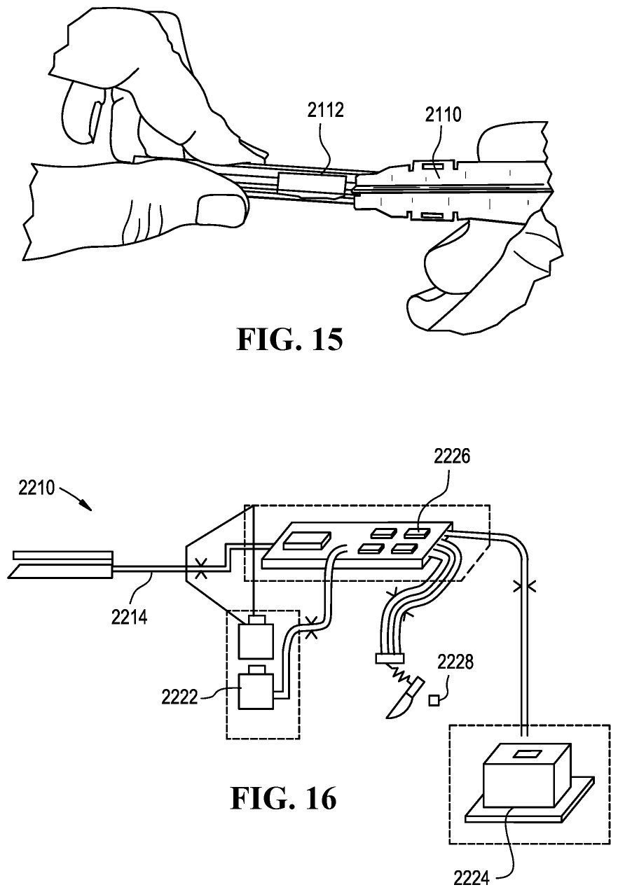

Before the disposal and/or reclamation process can occur, the contents of the smart packaging system 10, such as the surgical instrument 14, may need to be disassembled or separated into components and sub-components, depending upon the nature of those components and sub-components (e.g., non-recyclable versus recyclable components) as their nature may lead to differing disposal and/or reclamation processes, as explained herein. In some variations, the smart packaging system 10 can include one or more specialty fixtures, such as certain proprietary screws and the like, and associated tools to aid in reclamation of specific components. Certain components to be reclaimed can be fastened to the surgical instrument 14, for example, and the associated tool can be used to easily remove the component for separation. An example can be seen in FIG. 15, which features a simple pry bar 2110 to allow the removal of a circuit board 2112. The pry bar 2110 can be inserted into a specially-design slot and levered to disengage the circuit board 2112 from a surrounding component. In some variations, the packaging itself can be disassembled into a tool like the pry bar 2110. The packaging 12 can feature ribs that can support the strength of the packaging during transit and handling, and during disassembly, the ribs can double as the pry bar 2110.

In some variations, the packaging 12 can include tools that appear after components are removed from the packaging or a compartment of the packaging is opened. For example, a certain kind of tool can be revealed when a seam is pulled, a surgical tool is removed from its place, or a tab is pulled. In other variations, components of the smart packaging system 10 that include one or more components to be reclaimed can be packaged using packaging included within the smart packaging system 10, and the packaging 12 and components can be sent to an affiliated site specializing in disassembly and reclamation of the components, thereby placing the task on the affiliated site.

The smart packaging system 10 can also measure or evaluate portions of the surgical instrument 14 to determine their ability to be reclaimed, as well as to determine the associated path of disposal, based on various factors. For example, based on a determined geopolitical location, availability of waste management procedures, components of the smart packaging system 10, the smart packaging system can determine whether or not a certain component can be reclaimed. This indication can be provided to a user using any of the methods described herein.

Disposal and reclamation procedures can involve hazardous materials, including electrical components and biohazardous materials. Procedures for electrical components can involve the inclusion of materials and features in the smart packaging system 10 to minimize potential dangers involved. These can include insulated surfaces to house the materials, as well as electrical tape to bind and secure components as necessary. Procedures for biohazardous components can involve containing the components and preventing cross-contamination during the various post-operational procedures. For example, storage bags and/or bleach-soaked rags can be included in the smart packaging system 10 for containment of the components as well as for wiping down surfaces. In some variations, the storage bags can be made of a material having an anti-microbial property to prevent the spreading of certain materials. The bags can also contain absorbent materials to capture fluids placed within them. In some variations, the smart packaging system 10 can include blade covers and sharps covers to prevent the breach of containers of biohazardous materials, as well as to prevent accidental harm from the blades or sharps. To contain the biohazardous materials, the contaminated components can be broken down and contained within the sterile field.

Disposal and reclamation procedures may occur in an operating room without the oversight of the HUB 50 or similar system. In these circumstances, and where known, the smart packaging system 10 can include features to aid in tracking the various components being disposed and reclaimed. For example, the packaging 12 used in disposal can include incremental serial numbers to easily coordinate several disposal smart packaging systems 10 at once. In another example, the component to be disposed or reclaimed could include a removable marker, like a sticker, that could be removed and moved elsewhere, such as on a tracking sheet, in order to monitor the status of the disposal or reclamation of a given component.

The smart packaging system 10 can provide selective and separate disposal pathways for components of the smart packaging system 10, as well as for sub-components of the components. This can include the identifying these components and sub-components and then providing methods for separating the smart packaging system 10 as needed to introduce the components and sub-components into their proper waste streams for proper waste management.

Certain components of the smart packaging system 10 may need to be disposed separately. These components can include batteries, electronic components, heavy metals, surgical instrument 14 components marked for disposal that are contaminated by anatomical waste, and surgical instrument 14 components marked for return that are contaminated by anatomical waste. For example, for surgical instrument 14 components marked for return that are contaminated by anatomical waste, specific additional contamination procedures may be required to sterilize the surgical instrument 14 components.

The smart packaging system 10 can be used to easily identify key aspects needed for the selective disposal and/or reclamation of the components in order for those components to be appropriately separated. In an example, a barcode can indicate which surgical instrument 14 needs to be reclaimed and returned. The barcode could be located on packaging of the smart packaging system 10. In another example, the HUB 50 and/or a visual identification system could be used to identify reclaimable components as well as the locations thereof. In another example, the smart packaging system 10 could contain components in a modular form. One sub-area of the smart packaging system 10 could contain a motor, another could contain a battery, etc. Staff in the operating room could assemble the surgical instrument 14 using the modular components, use the surgical instrument 14, and then disassemble the surgical instrument 14 back into modular components. The modular components to be reclaimed could be individually resealed in separate packaging 12. In another example, the surgical instrument 14 and/or the packaging could include a color coordination with the associated waste stream pathway. A separate schema could be used for devices or components thereof that are to be reclaimed.

Separation of the components can occur in a number of ways and may depend upon the identity of the identified component, such as whether the component is a battery, a circuit board, etc.

Procedures for batteries can vary depending on a number of factors. For example, if a battery is provided in a separate package than the surgical instrument 14, that same package could be used to separate and store the battery following disassembly. The same can be true for other components, where the package in which they were provided can double as a separation package following a surgical procedure. In some instances, special procedures may need to be followed in order to dispose of a battery. Battery disposal bins may not be universal, in which case sub-disposal instructions may be needed. If a battery is primarily lithium, for example, it can be sent to a proper recycling facility or disposal site. If a battery is rechargeable, separate disposal procedures may be needed. Proper separation of batteries can prevent them from being incinerated or improperly disposed, possibly in violation of local regulations.

Procedures for circuit boards can also vary depending on a number of factors. Shrouds can be removed to access the circuit boards to be disposed or reclaimed. Certain design choices in the devices and components of the smart packaging system 10 can be made in order to provide easier access and/or disassembly of the circuit board into components. For example, a circuit board can be located on a separate piece of plastic that is accessible through a battery cavity. Accessing the circuit board could be achieved through a simple pull-tab to rip out the circuit board. Wires could also be accessible to allow for easy severing to remove sensitive components. The smart packaging system 10 could include a bin or area that is included for storage and containment of loose components. If a circuit board includes breakaway attachments or components, the circuit board could be designed such that those attachments or components are affixed to the circuit board via a mechanical disconnect, such as via a snap-fit or equivalent to aid in disassembly. Flex circuits could be severed or made easily severable mid-circuit close to a distal-most attachment and away from certain components that may include heavy metals or precious metals. In the case where multiple circuit boards are used, the circuit boards could be interconnected with several flex circuits to allow for easy severability. The packaging 12 could also include indications to show where circuit boards are located, where disconnect points are located, which components should not be disconnected due to certain contents thereof, etc.

In another example, the circuit board could be made to lock to a battery once the battery is inserted into position. To remove the battery, the lock could make it impossible not to also remove the circuit board as well. Once both are removed, as simple switch or lever or equivalent, hidden prior to removal, could be toggled to cause the battery and circuit board to separate.

Instructions can be provided to a user in any form, at every step of separation, to walk them through the specifics of separation, based on the components to be separated within the smart packaging system 10, as well as based on the relevant waste management procedures. Further, passive guidelines could be provided as well, such as an appropriately-shaped slot or container for each component to be separated out. If a certain slot or container exists, a user can be passively informed that it must be filled by some to-be-separated component during a separation procedure.

In some variations, separation of components may not be required by a user and instead, the entire product, package, sub-component, etc. could be returned as-is. Portions of the smart packaging system 10, such as certain packaging 12, could be reused to return the components of the smart packaging system 10. This packaging 12 could include plastic bags for biologically-contaminated products, etc. Further, while packaging may include separate sub-areas housing the necessary components of the smart packaging system 10 (i.e., there is a first space that is molded to fit the surgical instrument 14, there are second and third spaces that are molded to fit accessories), the packaging 12 could be made separable to remove the sub-areas to turn the packaging 12 into a single box or container. A user could then place the reclaimed components into the single box or container with no regard for organization.

Once the components are separated, they can follow their designated pathway toward disposal or reclamation. For example, certain components may be marked for incineration and they can be sent to an incinerator. Other components may be marked for a landfill and they can be sent to a landfill. For components marked for reclamation, procedures can vary based on the components. Metal and plastic components can be ground and melted at certain temperatures to separate out individual components, such as precious metals. If certain expensive plastics are to be reclaimed, or aggregate materials are to be reclaimed, such that the grinding/melting procedures are ineffective, there can be some visual indicator for user located on the component (or included in the instructions) that informs a user of the appropriate specific reclamation pathway for those special materials. This could involve simply shipping the special materials to a site specializing in the relevant reclamation.

Standard procedures can also exist to deal with more traditional waste materials, such as sharps. For example, needles used with sutures and knives can be separated out and then disposed in proper bins, according to specific healthcare procedures.

The HUB 50 and/or smart packaging system 10 can provide notifications related to waste management procedures. The HUB 50 can be aware of proper waste management procedures and becomes aware that some part of the proper waste management procedures are not being carried out correctly, the HUB 50 can notify a user using any of the methods described herein. For example, the notification to a user could indicate that a component related to the smart packaging system 10, as well as any other component of which the HUB 50 is generally aware, improperly exited a sterile field.

An example of the identification and coordination of components and sub-components of a surgical instrument 2214, part of an exemplary smart packaging system 2210, is depicted in FIG. 16. The smart packaging system 2210 can be generally equivalent to any variation of smart packaging system described herein, including the smart packaging system 10, and the same can be said of the surgical instrument 2214 with respect to any surgical instrument described herein, including the surgical instrument 14. The surgical instrument 2214 can be sub-divided by area of interest, including identifying a motor 2222, a battery 2224, a circuit board 2226, and a magnet 2228. The members of each identified area of interest can be associated with a certain disposal pathway. While some members may have the same disposal pathway, each can have a separate disposal pathway. For example, in a first area of interest, associated with the motor 2222, magnets, cooper coil, rare earth metals, etc. located within the motor 2222 could be harvest for recycling and/or they could be selected for disposal according to certain local regulations. In a second area of interest, associated with the battery 2224, the battery 2224 may need to be discharged, isolated in the event that it contains certain precious or toxic materials, and cannot be transported via certain modes of transit, such as a plane. These requirements could affect the disposal pathway for those materials. For example, European regulations may prohibit the disposal of batteries containing some hazardous substances, and they may also establish relevant schema for the collection and recycling of batteries. In a third area of interest, associated with the circuit board 2226, contents of the circuit board 2226 such as silicon, heavy metals, potentially precious metals, etc., could require separate disposal paths depending upon the relevant local geopolitical regulations. In a fourth area of interest, associated with the magnet 2228, disposal and disassembly could vary depending upon relevant local geopolitical regulations.

The procedures associated with FIG. 16 are exemplary and can vary depending upon many variables, including relevant local geopolitical regulations, the components of the surgical system 2214, available procedures, and more, as discussed herein.

The smart packaging system 10 can, based upon a current geopolitical location of the smart packaging system, selectively alter, engage, and disengage functions of the smart packaging system 10 in order to comply with relevant local regulatory schemes.

The smart packaging system 10 can make a determination related to the appropriateness of its components and capabilities for a given geopolitical location, based on the regulator scheme of that geopolitical location. The smart packaging system 10 can provide information related to its viability with the location to user for their assessment, or the smart packaging system 10 can compare its current location to a known “usability zone” for a given aspect of itself. For example, EU smoke evacuators require a HEPA filter to prevent aersolization of cancer cells into the air breathed by the healthcare providers, which is not the case for all jurisdictions. A smart packaging system 10 that contains a smoke evacuator and knows it contains a smoke evacuator can compare this known information and information about its current geopolitical location to a list or database of regulatory information in order to make the determination. The smart packaging system 10 can provide a message to a user informing them of this regulatory requirement. In another example, the smart packaging system 10 can inform a user if a drug is not certified for treatment or use within the geopolitical location.

In some variations, individual facility rules may be important to operations of the smart packaging system 10. If a facility is known ahead of time, instructions and specific regulations relevant to the individual facility can be pre-loaded into the smart packaging system 10.

In some variations, the smart packaging system 10 can collect and store data. Based on the current geopolitical location of the smart packaging system 10, the data collection capabilities of the smart packaging device and/or the data being collected by vary to comply with local rules.

In some instances, certain components and/or capabilities of the smart packaging system 10 may be outright prohibited from entering a certain geopolitical location. If it is determined that the smart packaging system 10 has ended upon in such a location or is about to enter such a location, the display 22 could display a warning indicating the prohibition (e.g., “PRODUCT BANNED IN CHINA”). In another example, the surgical instrument 14 may contain a battery that is designed to deploy a compound (e.g., bovine pericardium) that may not be acceptable in the jurisdiction. The display 22 can be configured to change to a message that includes, for example, “CONTAINS BOVINE PERICARDIUM.” This message can specify the offending component or contents of the smart packaging system. In another example, in 1985, China banned the importation of products derived from blood and/or plasma. If the smart packaging system 10 contains such products, the display 22 can provide a message, similar to those described above, that explains that there is a prohibited substance contained within the smart packaging system 10. This determination can be based upon FDA, CE, UL, or other approvals.

Generally, the smart packaging system 10 can make a determination of the appropriateness of its transportation into the given geopolitical region and then provide that information as needed.

In instances where the evaluation results in a determination that one or more operative capabilities of the smart packaging system are non-compliant with the set of local rules of the jurisdiction, the smart packaging system 10 can alter and/or deactivate—or receive a set of instructions from an external system to alter and/or deactivate—the non-compliant operative capabilities in order for the smart packaging system 10 to comply with the set of local rules. For example, the smart packaging system 10 can be configured to collect data during its transportation and usage, in various locations such as warehouses, hospitals, and operating rooms. Aspects of this data collection may not be permitted in the jurisdiction as a result of local privacy laws, such as HIPAA, GDPR, etc. In such jurisdictions, the ability of the smart packaging system 10 to collect the impermissible data can be deactivated in order for the device to operate according to the local privacy laws of the jurisdiction. In some cases, limiting or curtailing the operative capabilities may occur as a result of the mismatch between companion systems, such as when a certain component is not yet approved for use in the given geopolitical location.

In some embodiments, the controller 13 of the smart packaging system 10, or any equivalent storage of the smart packaging system 10 can have certain decision-making capabilities integrated therein.

In some embodiments, the decision-making capabilities can include an integration of multi-level decision-making within the smart packaging system 10 generally, and in the packaging display 22 using hierarchical organization techniques. These techniques can include the selection of components driven by physician preference, and/or the selection of components driven by component availability.

When one or more smart packaging systems 10, still in their packaging 12, enter an OR, the HUB 50 can communicate with the smart packaging systems 10 and create a list of the smart packaging systems 10, their contents, their characteristics, additional known information, etc., and the HUB 50 can also receive information related to the procedure to be performed within the OR, such as specific patient biometrics, type of procedure, surgeon identity, surgeon preferences related to the smart packaging systems, OR capabilities, etc. Based on this information, the HUB 50 can evaluate information related to interactions that can occur between the various systems in the OR and the procedure to be performed.

In an example, the HUB 50 can, based on stored data related to surgical operations, run through scenarios for how the scheduled procedure could be performed using the systems detected within the OR. The HUB 50 could inform the OR staff if there are unnecessary systems include in the OR, as well as if there are missing systems that should be added to the OR for the given procedure. If there are missing systems that should be added to the OR for the given procedure, the HUB 50 can provide a list of possible alternatives, in order of hierarchical preference, with or without receiving a prompt from a user. In variations in which a prompt from a user is received, the prompt could inform the HUB 50 that there is a lack of availability for the desired system, thus prompting the HUB 50 to provide the list of alternatives.

In another example, the HUB 50 can recognize that a smart packaging system 10 is included in the OR that is incompatible with one or more other systems in the OR. The HUB 50 can notify the OR staff of this incompatibility.

In another example, the HUB 50 can cross-check devices logged within the OR against the capabilities of the OR and determine whether there is an issue with compatibility. If a device is added to the OR that requires the use of additional equipment not located in the current OR, such as a large piece of machinery that is not easily movable to the OR, the HUB 50 can notify the OR staff of this missing machinery and provide a recommendation for another OR that would possess that necessary machinery.

As introduced above, patient data can be used to determine compatibility with smart packaging systems 10. When a smart packaging system 10 enters an OR space, the smart packaging system's 10 capabilities, contents, and other details can be analyzed against relevant patient biometrics/data in order to provide information related to optimal device selection and usage.

More specifically, a determination can be made as to device compatibility. If a patient has undergone prior surgical procedures, that information can be used in a current surgical procedure to determine device compatibility. For example, if the patient's body contains metal from a previous surgery and the current surgery plans to involve an RF device, a notification can be provided of an incompatible use, possibly arising from a short or other malfunction as a result of the interaction between the RF device and the metal. More local implications can also be considered, depending on the surgical instrument 14 being used. If the surgical instrument 14 is a harmonic tool, a metal detector on a tip thereof can audibly alarm a user if metal is nearby. Tissue impedance could be impacted by metal, possibly resulting in harm to the patient and/or to the surgical instrument 14, and such a scenario should be flagged. If the surgical instrument 14 is a stapler, detection of the end of a segment for stapling can result in the stapler being prevented from further stapling an undesirable area. In some aspects, the surgical instrument 14 can adapt to an adverse event detected. For example, the surgical instrument 14 could employ an adaptive situation algorithm that adjusts power delivered to the surgical instrument 14, such as to drive a staple or incise tissue, when an impacting material is detected in close proximity (i.e., metal is nearby so the smart device shortens a drive stroke to not hit the metal).

Further, patient specific tissue can alter device behavior and compatibility. Tissue which has been radiated can be tougher than other tissue, and device feedback could be handled differently. Thresholds in the surgical instrument 14 could be altered to account for the toughened tissue, such as increasing the power behind a drive stroke to make a certain incision that would normally require less power with normal tissue. Surgical instruments 1414 that can alter their behavior can also be configured to receive input from an external system, such as the HUB 50 or notes from an operator that account for the changes being made in response to the patient tissue. Such changes could impact the lifecycle of the surgical instrument 14, especially if a larger demand is being asked of the surgical instrument 14 as a result of the changes.

In another example, certain ablation systems can be used only with tumors of a certain size (e.g., 10 cm) or smaller. If a tumor is detected that exceeds this threshold, the surgical instrument 14 can warn a user and give a the user a choice to override the warning. The size of the tumor could be communicated by the HUB 50 to the smart packaging system 10 as a result of a scan. The display 22 and/or display 22 can update even before the smart packaging system 10 is opened to reflect a possible incompatibility issue.

In another example, if a certain task in a surgical procedure requires a power output that is much lower than the capability of the surgical instrument 14, a warning could be displayed on the display 22 or the display 22 of the smart packaging system 10 informing a user of the possible danger associated with using the surgical instrument 14 at its normal power level.

Alternatives to the surgical instrument 14 can be provided based on cost, stock, or healthcare facility guidelines. For a given procedure, once all smart packaging systems 10 have entered an operating room, their capabilities can be compared by the HUB 50 against the requirements of the procedure itself. Based on this comparison, the HUB 50 can provide recommendations for more efficient and/or optical devices that can be used in place of what is currently found in the operating room. For example, the HUB 50 recognizes that an energy device was pulled for a procedure and also recognizes that, based on the planned procedure, a monopolar or bipolar energy device is traditionally used. The HUB 50 reviews the pulled energy device, determines that the pulled device is insufficient, and then reviews the other devices for potential alternatives. After determining that there are no alternatives, the HUB 50 can search inventory at the healthcare facility and make a request for a capable alternative. A user can be notified of the request and a capable device can be delivered to the operating room.

In some variations, the HUB 50 can determine that a selection of products pulled for a procedure exceeds reimbursement limits. Based on the total cost, as presented in a fiat currency, operating room time, number of staff, or another metric, the HUB 50 can make alternative recommendations. Further, the smart packaging systems 10 can display their own unit cost for a user, or provide that information directly to the HUB 50 without the HUB 50 needing to conduct a tally. Alternatives can be ranked based on cost, historical operations data, and any other desirable metric.

To assist with decision making by the smart packaging system 10, metadata can be included in the smart packaging system 10 to provide context for irregular usage. Such context can arise with conformational flagging and interrogation flagging.

With conformational flagging, irregular data can be received by the smart packaging system 10, the HUB 50, or another device that appears to be facially correct but which appears odd in context. A notification can be provided to a user the receiving system that does not require confirmation by the user for further operations to proceed. For example, if a doctor selects a surgical instrument 14 that is not normally used for a procedure, confirmation can be received once that this was an intentional choice, and then every time in the future that the system would potentially flag some aspect of the surgical instrument 14, an override can be triggered that recognizes that this surgical instrument 14 selection was intentional and that the surgical instrument 14 is otherwise behaving normally. Such an override can also come into play where an alternative surgical instrument 14 is selected as a result of a stock shortage of the preferred surgical instrument 14.

With interrogation flagging, user input can be solicited for further decision making upon receipt of irregular data. For example, when an incorrect surgical instrument 14 type is detected, user confirmation can be required before operations are permitted to proceed. The smart packaging system 10 can generate an alert indicating the incorrect surgical instrument 14 type, and confirmation can be solicited from a user. In another application, when surgical instrument 14 having an incorrect device length is selected for a procedure, an override scenario can be implemented based on historical data concerning the procedure. For example, if operations occurring in a surgical procedure are dependent upon a specific surgical instrument 14 size, those operations can be adjusted in view of a new surgical instrument 14 size.

In some scenarios, multiple device options can be sent to an operating room based on historic data for user selection. Whichever surgical instrument 14 is chosen, stock is notified of the surgical instrument 14 to be returned to minimize user interaction.

When an insufficient smart packaging system 10 has been pulled, the HUB 50 and/or the smart packaging system 10 itself can react accordingly. For example, if an aspect of a surgical procedure or of the patient has flagged that additional surgical instruments 14 may be required to complete the surgical procedure in view of the aspect, interrogation of a user can confirm that the flagging was correct and that additional surgical instruments 14 are in fact needed. The interrogation can be handled via any means of communicating with a user to confirm that additional surgical instruments 14 are indeed needed and can be requested from a stockroom. In another example, the HUB 50 can recognize that for a given procedure, six staple cartridges are usually pulled. However, based on patient biometrics, an additional staple cartridge needs to be pulled with typical patients of this size. The HUB 50 can place the request for an additional cartridge and a user can be notified. Similarly, if six staple cartridges are pulled for a surgical procedure but the HUB 50 detects that, based on historical data of similar surgical procedures, patients of this size normally need just four staple cartridges, the user can be notified of the potential for excess.

Responses to the various inputs received from users can be recorded to inform future suggestions by the smart packaging system 10 and the HUB 50. These can include incremental alterations by suggesting similar products, exceptions based on surgeon or facility, and repeated confirmation of specific responses based on an answer (e.g., the HUB 50 flags a surgical instrument 14 three times and a user refuses each time; the HUB 50 can adapt and not flag the surgical instrument 14 a fourth time).

In some embodiments, the smart packaging system 10 and/or the HUB 50 can determine compatibility between their own capacities and other devices, including other smart packaging systems 10. The smart packaging system 10 can provide a notification of possible interactions, both acceptable and adverse, between itself and other devices, as well as between other devices. This information can be especially beneficial in the context of a surgical procedure.

In some variations, the smart packaging system 10 can provide a notification of potential interacts with other equipment that may be dangerous and/or harmful to patients, personnel, and equipment.

As explained herein, both the HUB 50 and the smart packaging system 10 possess the ability to receive and transmit information with other systems. Based on this transfer of information, the HUB 50 and the smart packaging system 10 can assess potential interactions between systems and devices. When an incompatible combination is observed, a user can be notified, such as via the display 22 or other notification. Further, the system that detected the incompatibility can transmit information to the HUB 50 (unless the HUB 50 is the detecting system), which can then transmit instructions to affected smart packaging systems 10 and devices to alter or cease operative capabilities to prevent an adverse interaction from taking place.

For example, certain surgical staples can include a magnesium-based coating. Exposure to high heat can damage the coating and jeopardize the integrity of the staples. In another example, if a CT device, such as a C-arm, is being used to scan a patient who possesses a pacemaker, the HUB 50 can notify a user of a possible adverse reaction and/or instruct the C-arm to alter operations so as to not interfere with the pacemaker's operative functions.

In some variations, a risk-based assessment of the combination can be performed, and a corresponding notification can be provided. If the interaction is deemed to be potentially catastrophic, the HUB 50 can communicate this risk to other equipment to prevent that equipment from activating and resulting in possible harm. For less catastrophic events, a user can be notified of a potentially adverse interaction, and a confirmation can be required from the user before normal operative functions are enabled. For minor interactions, a non-actionable warning can be displayed for sent to a user to inform them of the interaction.

In an example, new generation reload cartridges for surgical staplers can be designed with backwards compatibility in mind, but this is not always the case. In certain instances, new generation reloads may be incompatible with certain surgical staplers, and an attempt to combine the reloads with the ineffective surgical staplers can result in damage to one or both components. An indication to a user prior to the interaction could resolve the potentially dangerous situation.

In some embodiments, the smart packaging system 10 and/or the HUB 50 can contribute to the adaptation of surgical procedures as a result of specific limitations of that procedure, such as alternative surgical instruments 14, patient need, etc. The details of a surgical procedure can be imported to the HUB 50, and the HUB 50 can flag interactions between surgical instruments 14, accessories, and devices that should be avoided, as well as combinations that should be used together.

When the HUB 50 flags interactions that should be avoided, a user can override the flag and proceed anyway, as explained above. When this occurs, certain data may be inaccurate or corrupted by the flagged interaction. To avoid presenting the inaccurate data, no data can be provided in these scenarios. For example, if an electrocautery device is used on a patient currently being monitored by an EKG, the electrocautery device can interfere with the readouts of the EKG. Such an adverse interaction between the electrocautery device and EKG can be flagged by the HUB 50, but in the event that a user proceeds anyway, the readout from the EKG can be prevented from being displayed to avoid presenting misleading readouts while the electrocautery device is in use. This kind of adverse interaction, especially related to electrical interference of devices, can result in completely wrong data.

In some variations, an impact of the interaction on the data collection could be measured, where possible, and a notification could be provided to a user of the impact and the degree of the impact. This can be done by measuring the same source of data without the presence of the interfering device and comparing it to the data when the interfering device is present. Based on historical trends, past data measurements, machine learning, secondary sources, etc., the measured impact of the interfering device can be provided to a user. Further, a bit error rate and/or cyclic redundancy check (CRC) error detection code can be used to determine the impact of the interference. In some aspects, a suggested change to reduce or minimize the impact of the device could be provided by the HUB 50, e.g., adjusting settings of the device, repositioning the device, etc.

When the HUB 50 flags interactions that should be promoted, these interactions can be provided to a user to assist in a surgical procedure. In an example, during a colorectal surgery in the context of a circular stapler anastomosis through previous linear staple lines, the HUB 50 can recommend certain device combinations. These combinations can include a recommendation for a stapler (e.g., powered versus mechanical) to best complete the procedure based on the plan for the anastomosis. The powered stapler, an example of a possible smart surgical instrument 14, could suggest staple alignment by sensing a staple location (e.g., through impedance, etc.) and suggesting a reorientation. For example, if, after a recommendation, realignment occurs that is unsatisfactory, a new recommendation could be provided by the powered stapler. If the procedure continues anyway, future aspects of the procedure could be adjusted based on the unsatisfactory realignment. In another example involving a circular stapler, preferred performance of the circular stapler may require that a trocar used with the stapler be pushed through a center of a previous linear transection stapler line. When aligned, a staple height should be adjusted based on the thickness or other properties of the tissue in the relevant location. If a user does not properly align the circular stapler or adjust the staple height, later steps in the procedure involving the operation could be adapted (e.g., rate of firing, wait periods between firing, etc.) in order to compensate for the user's failure to make the alignments and/or adjustments. These adaptations could even cascade to later procedure steps such as pressure testing of a created anastomosis, mobilization, etc., all of which could be governed by the HUB 50.

In some variations, the HUB 50 and/or the smart packaging system 10 are able to recognize if a device is not optimal for a given surgical procedure, in which case, the HUB 50 and/or the smart packaging system 10 can recommend an alternative device. For example, if an older device is detected that is missing certain features beneficial to the surgical procedure. If an alternative device is not used and incompatibility can impact a procedural outcome, a user can be notified. If the procedure continues with older device, the HUB 50 and/or the smart packaging system 10 can notify a user of certain procedural steps that may be impacted by the incompatibility, and adjustments can be provided to compensate for the incompatibility at the impacted procedural steps. For example, adjusting a current set point of a harmonic device to deliver less power, or shifting a frequency range of a bipolar energy device to reduce potentially adverse interactions with other devices.

In some embodiments, the smart packaging system 10 and/or the HUB 50 can suggest complimentary products or preferred alternatives based on products currently selected for a surgical procedure. For example, a recommendation could include a newer model of a selected surgical instrument 14 that can result in greater efficiency in a surgical procedure. The suggestions can be provided before, during, or after a surgical procedure, and the suggestions can affect future surgical procedures. Suggestions provided before a surgical procedure can rely on past data and recommendations to flag alternatives before the surgical procedure begins. For example, if a previous recommendation was heeded for a prior procedure, the same recommendation can be made prior to the beginning of a similar procedure. Suggestions provided during a procedure can be provided by the HUB 50 in real time based on procedure status. During a procedure when a recommendation is made, the recommendation can also include an estimated time to collect the recommended alternative, and the HUB 50 can solicit a user for input as to whether they would like to tag this recommendation for future surgical procedures so that it becomes a default option for those future surgical procedures. Suggestions provided after a procedure can be based on observed trends and data collected from past procedures. Recommendations can generally take into account specific procedures and usage data of the healthcare facility.

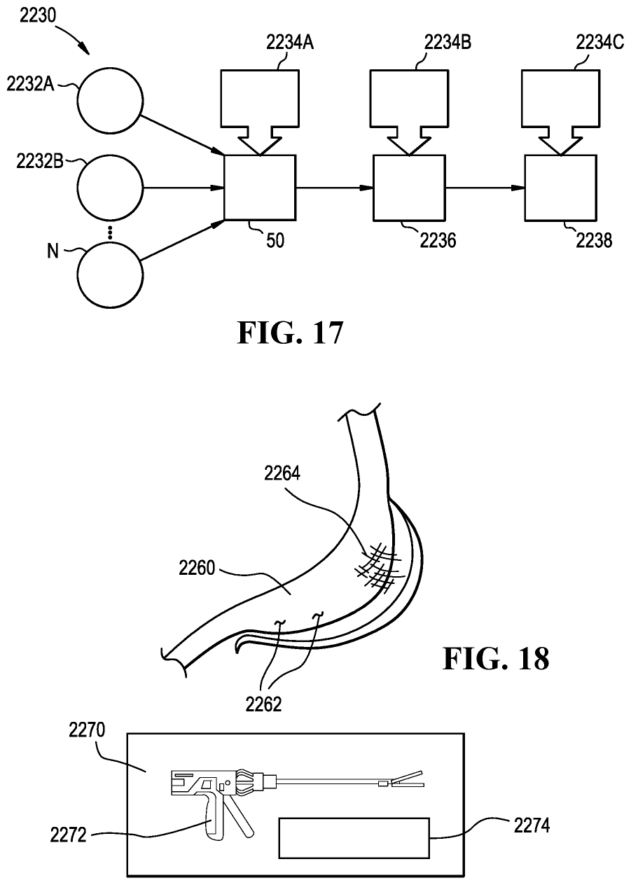

As seen in FIG. 17, a general process 2230 for recommendations is depicted, which details the way in which specific smart packaging systems can be selected for use in a procedure. One or more smart packaging systems 2232A, 2232B . . . N can be in logged by the HUB 50, as explained herein, along with additional information 2234A from the other sources, such as information about the facility logging the systems, information concerning the devices themselves, including interaction information, updated procedures, recall notices, etc. When a recommendation is required, a sub-set of the logged systems 2236 can be provided, which can be informed by additional information 2234B. This additional information 2234B can include information such as availability, location of the systems within the facility, patient information, procedure demands, etc. Once the sub-set 2236 has been provided, a final result or selection 2238 can be made in view of final information 2234C. This final information could include information such as surgeon preference, negative interactions with other selected devices for the given procedure, and other information that can disqualify potentially viable selections.

In an example, use of a harmonic foot petal can be an optional accessory for a surgical procedure. A recommendation can be made to a surgeon to suggest the use of the harmonic foot petal, and this recommendation can be based on a number of factors, including: past preference of the surgeon, ease of use with other devices pulled for the surgical procedure, prevalence of use across the healthcare facility, and the nature of the specific procedure.

In some aspects, an OR configuration can be adjusted based on devices pulled for a surgical procedure. For example, based on a length of a pulled device, the HUB 50 can recommend a specific surgical cart, an intentionally-sized operating table, additional accessories, etc. In another example, if multiple energy devices are pulled for a surgical procedure, the HUB 50 can recommend the use of two generators in the OR to compensate for the pulled devices. In another example, if an older insufflator is available in the OR, a newer trocar can be suggested to optimize a seal integrity between the systems. In another example, if an older smoke evacuator is present in the OR, recommendations can be made for newer devices that produce less smoke, or newer pumps can be recommended that are more efficient so that the older smoke evacuator remains effective.

In another example, multiple monopolar tips can be used with the surgical instrument 14, and a suggestion for a specific monopolar tip can be provided for a given surgical procedure where that specific monopolar tip would be more effective. This suggestion can be made in view of alternative monopolar tips that would be more effective only in specific scenarios, and the alternative monopoloar tips would not need to be pulled for that surgical procedure at all.

In another example, an incomplete set of smart packaging systems 10 and other devices and components can be detected by the HUB 50 for a given surgical procedure, and the missing elements can be located by the HUB 50. This kind of evaluation and detection by the HUB 50 can occur prior to a surgical procedure, as explained above, and it can also occur after a surgical procedure to prevent components and objects being retained in a patient—a known issue. Retained objects—objects unintentionally left in a patient post-op—can result in dangerous complications like perforations, infections, and obstructions.

In some variations, compatibility between two surgical instruments 14, components, accessories, etc. can be determined based on a geometric aspect of one of the items. For example, if a surgical stapler is compatible with a 45 mm staple cartridge, but not a 60 mm staple cartridge, a notification can be provided to a user if a 60 mm staple cartridge is pulled for a surgical procedure.

In some variations, as explained above, the smart packaging system 10 can include a readable medium 20. The readable medium 20 can link to manufacturing information of the surgical instrument 14 and/or to a central database of device information, generally, to support recommendations based on cross-references to other devices and possible alternatives. For example, a smart packaging system 10 containing an endocutter can be scanned before opening the smart packaging system 10. The HUB 50 can receive the data obtained from the scan, which can link the HUB to a database that contains manufacturing parameters for the endocutter, including force-to-fire, cut line length, articulation angle range, etc. The HUB 50 can provide a notification indicating that the endocutter is best suited for a certain tissue thickness, at a certain location on a patient, and other information based on the manufacturing parameters of the endocutter. In a similar example with a harmonic device, manufacturing parameters can include blade length and excursion (correlating to thermal spread potential).

In FIG. 18, a simplified view of a patient's stomach 2260 is depicted. The stomach 2260 includes staples 2262 from a previous procedure, as well as scar tissue 2264. Both the staples 2262 and the scar tissue 2264 can limit the kind of surgical device that can be used in subsequent procedures, as well as can limit operations that are able to be successfully performed by future devices. This information can be included in patient data, of which the HUB 50 and/or smart packaging systems are aware. In a future procedure, if a smart packaging system 2270 2270 containing a surgical instrument 2272 capable of delivering bipolar energy for artery dissection is brought to an operating room in preparation for a procedure involving the patient's stomach 2260, an attempted use of the surgical instrument 2272's bipolar capabilities could interact poorly with the staples 2262 and potentially harm the patient. The smart packaging system 2270 can, prior to being opened, determine that this risk is present and provide a message to surgical personnel warning them of the risk and/or incompatibility. The message could be provided in a variety of ways, including via a display 2274 (e.g., a smart label or e-ink label).

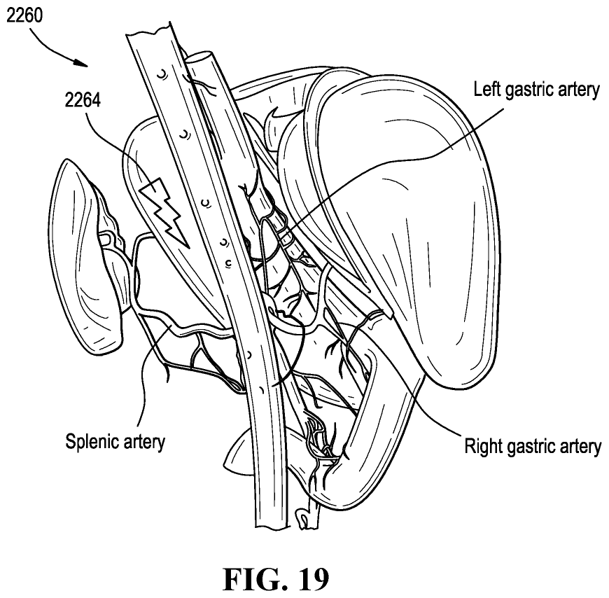

In a gastric sleeve revision a patient's stomach remodels itself causing the stomach tissue to become thick and stiff. It can be challenging to restaple or re-sleeve a line over top of a previous sleeve staple line as a result of the remodeling. Some staplers are better at handling this challenge than others. For example, certain powered surgical staplers (e.g., carrying the Echelon® name) have an I-beam, and would be much more capable than a non-I-beam stapler at accomplishing this task. In FIG. 19, the stomach 2260 with the remodeled tissue 2264 is depicted, including various structures located surrounding the stomach 2260. The stomach 2260 could be displayed on a screen or GUI located within an operating room prior to a surgery, along with information surrounding the relevant patient information that may impact a present procedure. Generally, the smart packaging system 10 and/or the HUB 50 could be made aware of such a previous stomach procedure, and if a non-I-beam stapler is selected, notice could be provided that a better option exists for the procedure.

In some embodiments, the smart packaging system can include features that assist in maximizing storage and containment efficiency of the smart packaging system itself, as well as of accessories, devices, and equipment contained within the smart packaging system. The smart packaging system 2310 can include a packaging 2312 containing a surgical instrument 2314. Generally, the smart packaging system 2310 can be similar to the smart packaging system 10, and features of the smart packaging system 10 can be applicable to the smart packaging system 2310. The packaging 2312 of the smart packaging system 2310 can include physical characteristics that facilitate functions beyond those necessary for transit from the manufacturing site to a usage site. The physical characteristics can include features which allow for the surgical instrument 2314 (or components thereof) to be stored and returned to a producer follow usage of the surgical instrument 14, as well as to be protected during that storage and return process.

The packaging 2312 of the smart packaging system 2310 can include one or more physical aspects that assist in the control and/or storage of components of the surgical instrument 2314, and/or secondary components, within an operating room during usage of the surgical instrument 2314 in a surgical procedure. In this way, the packaging 2312 can function beyond mere protection of the surgical instrument 2314 during shipping and handling of the surgical instrument 2314. It instead can function beyond that initial transit period.

In some aspects, the packaging 2312 can be used for product management within the operating room during the surgical procedure. For example, an aspect of the packaging 2312 can assist in cord management.

In other aspects, the packaging 2312 can include selectively-openable packaging which can be opened at key points during the surgical procedure when the contents of the selectively-openable packaging are required. Rather than storing all of its contents in a single location within the packaging 2312, the components can be divvied up based upon the procedure in question. The selectively-openable packaging can be made selectively openable in a number of ways.

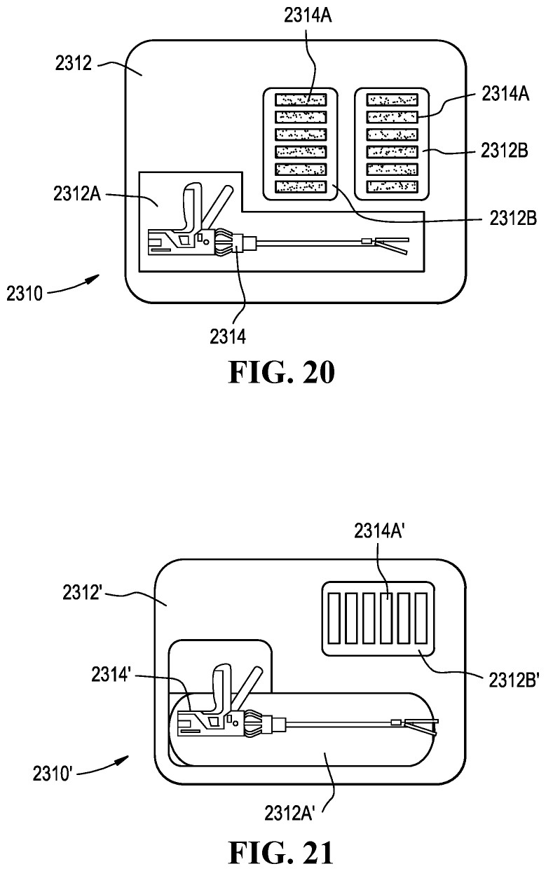

In some variations, the packaging 2312 can feature a main section 2312A to hold a surgical instrument 2314, as explained above, and at least one sub-section 2312B to retain additional components, such as accessories 2314A usable with the surgical instrument 2314. Based on a size of the surgical instrument 2314, the smart packaging system 2310 can feature packaging 2312 having a different layout, including differently-sized main sections 2312A and a differently-sized at least one sub-section 2312B. Examples can be seen in FIG. 20 and FIG. 21, which feature a similarly-sized packaging 2312 and 2312′ respectively. The packaging 2312 in FIG. 20 includes a smaller surgical instrument 2314 as compared to the surgical instrument 2314′ depicted in FIG. 21. As a result, the main section 2312A is smaller than the main section 2312A′. The smaller main section 2312A leaves room for a larger at least one sub-section 2312B, which can accommodate accessories 2314A either of greater size or of greater count than compared to the smaller at least one sub-section 2312B′, or leaves room for more than one at least one sub-section 2312B.

In one variation, where the timing of sub-processes in a surgical procedure are known, timers or time-keeping devices can be used to prevent access of a given selectively-openable package before a predetermined amount of time has elapsed after the commencement of the procedure. In another variation, the selectively-openable packaging can include one or more scannable markings per individual compartment of the selectively-openable packaging. The scannable markings could take various forms, such as a barcode or a QR code. Upon scanning the scannable markings, a signal could be sent to the container associated with the scannable marking that causes the container to open and provide access to contents stored within. In another variation, the containers could include one or more safety interlocks that are chained together. Each of the containers within the selectively-openable packaging could be ranked according to priority and/or order of use during the surgical procedure. In this arrangement, a certain container would be unopenable if a container of higher priority or which occurred earlier in the order of use had not been opened. For example, if the surgical instrument 14 is a device that includes a harmonic blade, the contents of a container within the selectively-openable packaging may include a blade cleaning solution that is recommended for use when the harmonic blade becomes too dirty. When the harmonic blade device is removed from the packaging 12, a secondary cover with a pull tab could prevent the blade cleaning solution from falling out of the package prematurely. In order to access the blade cleaning solution, the harmonic blade device must be removed from the packaging 12 and the pull tab attached to the secondary cover must be pulled to expose the cleaning solution. In this arrangement, the harmonic blade device has a higher priority than the blade cleaning solution, which is reflected within the structure of the outer packaging.

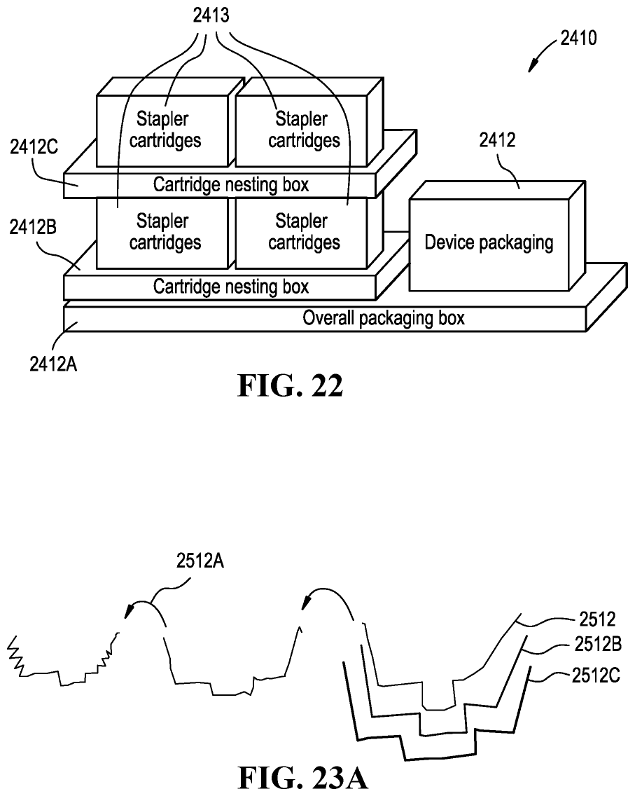

In an embodiment, a smart packaging system 2410 is provided, which can possess the same qualities as any of the smart packaging systems described herein. The smart packaging system 2410 can be shipped with sub-packaging 2413 arranged in a tiered or layered arrangement. An example arrangement of a smart packaging system 2410 can be seen in FIG. 22, which features a smart packaging system 2410 including a main packaging 2412 placed atop a base layers 2412A, and sub-packaging 2413 placed atop a secondary layer 2412B and a tertiary layer 2412C. The main packaging 2412 can contain a surgical instrument (not shown). The secondary and tertiary layers 2412B, 2412C only house the sub-packaging 2413, which can contain accessories for the surgical instrument 2414. For example, if the surgical instrument is a stapler, the accessories can be staple cartridges usable with the stapler. The sub-packaging 2413 on the tertiary layer 2412C can deliberately contain accessories used in a surgical procedure before the accessories contained in the sub-packaging 2413 on the secondary layer 2412B, so that the accessories used earlier are easier to access.

In some variations, the packaging 12 can include secondary-use packaging 12. The secondary-use packaging 12 can be an aspect or component of the packaging 12 that is retained within the packaging 12—or that is a physical characteristic of the packaging 12 itself—to assist in control or storage of one or more components of the smart packaging system 10 following usage of the smart packaging system 10. For example, the aspect could promote sustainability.

In one variation, the aspect of the outer packaging could facilitate the minimization of the physical footprint of the packaging 12. This minimization could be accomplished in a number of ways, including through self-compaction and/or user-enabled compaction. User-enabled compaction could include the use of outer packaging that includes a non-rigid interior packaging and a rigid outer layer to enable easy post-use separation of the outer and inner packaging, and simple disposal. The non-rigid interior packaging could be an impermeable covering, such as a kind made from a plastic, a polymer, or the like, and the rigid outer layer could include a rigid outer skeleton made from plastic, polymer, metal, or a combination thereof. The rigid outer skeleton could further be collapsible or separable into sub-components to further allow for easier disposal of the packaging 12.

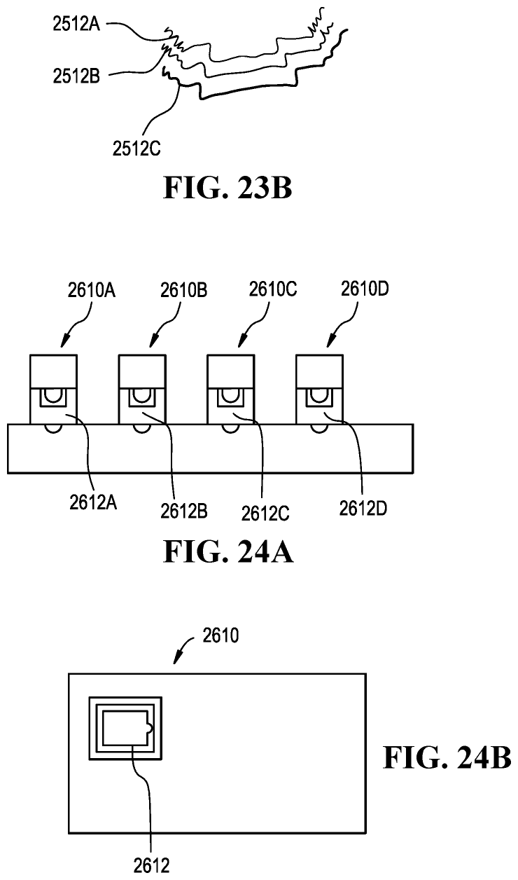

In addition to the inner packaging 12, minimization could be accomplished through the interlocking of sub-packages of the packaging 12. For example, the packaging 12 could be comprised of numerous interlocking panels that together form a complete outer package. Once the contents of the packaging 12 have been used, the panels could be separated from each other and allowed to lie flat. In another example, the packaging 12 could be made of a material that includes one or more foldable portions, such as corrugated cardboard. When compaction is desired, the packaging 12 could collapse along pre-determined points or folds and made to lie flat. In an example, seen in FIG. 23A and FIG. 23B, a variation of a packaging 2512 is depicted which can be used with the various smart packaging systems described herein. After the smart packaging system has been used, the various packaging 2512A-C included in the smart packaging system is collapsible and sized in a way that the packaging 2512 can be nested together to save on space, as depicted in FIG. 23B.

In addition packaging from the same smart packaging system being nestable, several smart packaging systems can include packaging designed to nest together. An example can be seen in FIG. 24A and FIG. 24B, which includes several smart packaging systems 2610A-D and their respective packaging 2612A-D. The packaging 2612A-D is depicted in a normal state in FIG. 24A and then in a nested state in FIG. 24B.

In another variation, the outer packaging could be designed such that the outer packaging remains intact after opening. Typically the outer packaging can be broken down or stacked inside other similar packages for waste management. If the packaging 12 were designed to be compactly handled without breaking being broken down, the packaging 12 could be reused for removal of the surgical instrument 14 from a surgical space. In such events, the label 22 could be used for tracking usage cycles of the contained surgical instrument 14. This information could be beneficial in circumstances including recoverable surgical instruments 14. With recoverable systems, the manufacturer can retrieve a surgical instrument 14, or component thereof, after each use, and the retrieved surgical instrument can be re-sterilized and resold. The packaging 14 used to retrieve the surgical instrument 14 could be reused and could track the number of times the surgical instrument 14 has been re-processed or used over its entire life.

In another variation, one or more components of the packaging 12 could be made recyclable in some way, such as being biodegradable, being reusable, and/or being recyclable. Additionally, components can be selected that are intentionally more eco-friendly. To improve reusability, the packaging 12 of the smart packaging system 10 could be made more durable, and components of the smart packaging system 10 can be chosen based on their durability. To improve recyclability, excess packaging materials in the smart packaging system 10 can be removed, and materials can be selected that are easily recyclable, such as pieces of interlocking cardboard, rather than various plastics. The smart packaging system 10 could also feature breakaway electronics to allow for increased severability among components that are placed into separate waste streams or are reclaimed entirely. This can make the breakdown process more efficient so that as many components are recycled as possible.

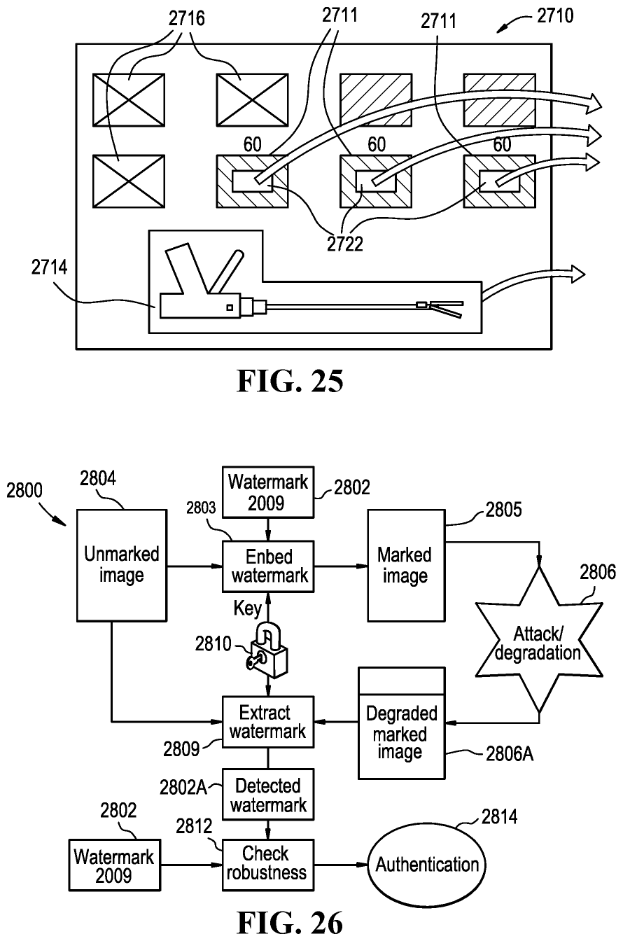

In another variation of the smart packaging system 10, depicted in FIG. 25, a smart packaging system 2710 is depicted that includes a plurality of nesting locations 2711 defined therein. These nesting locations 2711 can be located outside a sterile portion of the smart packaging system 2710, and can essentially consisting of a series of spaces to receive sub-packaging resting in predefined locations within the smart packaging system 2710. The sub-packaging 2713 can include attachments, add-ons, materials, etc., which are usable with the remainder of the smart packaging system 2710. For example, if the smart packaging system 2710 is a package for a stapler, the smart packaging system 2710 can include a stapler 2714 and staple reloads 2716 disposed within the nesting locations 2711, as well as additional materials. Each nesting location 2711 can include a dynamic label 2722, like the kind described herein, such that when a staple reload 2716 is removed from one of the nesting locations 2711, a corresponding dynamic label 2722 can display information related to the pulled reload 2716, including size, type, condition, and other information about the reload 2716 itself, as well as information concerning the greater procedure involving the reload 2716, including order of usage, purpose, and more. In this way, healthcare providers, such as the surgical team, can use this information to understand whether all components of a procedure are present and accounted for, and whether they are in good condition for the procedure.

The secondary-use packaging 12 can include aspects that help control the overall shape or configuration of the packaging 12. For example, the packaging 12 could be made to self-coil into cylindrical or pyramidal shapes to take up less of a footprint. The packaging 12 could also include various indents or features that enable other items to be stacked thereon in a secure manner.

The smart packaging system 10 can include improved packaging protection behavior and features. These can include active packaging features that react to various external stimuli. In one example, temperature logging by the smart packaging system 10 can ensure that the smart packaging system 10 and its contents were not exposed to temperatures which could potentially degrade the materials thereof. The smart packaging system 10 could be equipped with packaging made from materials that react to certain hot or cold temperatures to protect their contents.

In another example, the smart packaging system 10 can feature impact-reactive cushioning. For example, a corrugated design in the packaging can act as an absorption mechanism if the packaging is dropped or experiences forces that could potentially damage the contents thereof. In another example, the packaging can be made from a reactive material that, in the presence of UV light, darkens to protect the contents of the smart packaging system 10 from being exposed to the UV light, which is known to degrade many kinds of plastics.

Aspects of the packaging can enhance an authentication process for the components of the smart packaging system 10. The authentication process can occur prior to or during a surgical procedure in which one or more components of the smart packaging system 10 are used or are to be used, in order to act as extra security measure. Such an authentication process can prevent the use of a fraudulent, recalled, unsafe, or other kind of problematic product during the surgical procedure, thereby preventing patient and/or staff harm. To carry out the authentication process, the outer packaging of the smart packaging system 10 can include one or more verification markers, which can be detected by the HUB 50 or another system. Upon detection of the one or more verification markers, a determination can be made as to the authenticity of the smart packaging system in question.

The one or more verification markers can take on a number of forms, and may include markers that are disguised and/or entirely hidden from detection by a human eye (e.g., made in an ultraviolet ink), a hidden watermark, or a specific pattern of shapes contained within a larger, seemingly random pattern. For example, an array of dots could exist on the packaging, and the presence of a specific sub-set of dots (e.g., the sub-set of dots include a certain pattern of color, form, arrangement, etc.) can be detected and used to complete the authentication procedure.

The HUB 50 or another system can include a visual processing component or optical detector, such as a camera that can check for one or more verification marks on the packaging prior to use in a surgical procedure. These marks can indicate product authenticity, and can be disguised to be invisible or obscure to the naked eye. For example, infrared ink, unique patters, and more could all be computer readable, and yet a human would find it difficult or impossible to discern the marking. Further, these marks could be designed so that they only become visibility when the smart packaging system 10 is properly sterilized or re-sterilized. Further, separate systems could possess compatible marks, which could provide visual feedback of operable compatibility between the separate systems.

An example verification procedure 2800 for a watermark 2802 is depicted in FIG. 26. In FIG. 26, an unmarked image 2804 of a woman can be provided. A watermark 2802 including a depiction of a flag with the number “2009” and a circle with a line can be embedded at an embedding step 2803 within the unmarked image 2804 using a key 2810 so that it is now marked 2805. The marked image 2805 can be degraded or attacked at a degradation step 2806 resulting in a degraded marked image 2806A. This degredation could ensure the product has not been compromised. During a verification step 2808, the key 2810 can be provided to extract the watermark 2802A from the degraded image 2806A at an extraction step 2809, and a robustness check step 2812 can be used to compare the extracted watermark 2802A against the original watermark 2802. If there is sufficient similarity between the watermarks 2802, 2802A, at the robustness check step 2812 the verification procedure 2800 is complete and authentication 2814 is achieved.

Certain illustrative implementations have been described to provide an overall understanding of the principles of the structure, function, manufacture, and use of the systems, devices, and methods disclosed herein. One or more examples of these implementations have been illustrated in the accompanying drawings. Those skilled in the art will understand that the systems, devices, and methods specifically described herein and illustrated in the accompanying drawings are non-limiting illustrative implementations and that the scope of the present invention is defined solely by the claims. The features illustrated or described in connection with one illustrative implementation may be combined with the features of other implementations. Such modifications and variations are intended to be included within the scope of the present invention. Further, in the present disclosure, like-named components of the implementations generally have similar features, and thus within a particular implementation each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described implementations. Accordingly, the present application is not to be limited by what has been particularly shown and described, except as indicated by the appended claims. All publications and references cited herein are expressly incorporated by reference in their entirety.

What is claimed is:

1. A surgical system, comprising:
a manufacturer-sealed sterile surgical package containing a surgical instrument configured to be used in a surgical procedure;
at least one data processor disposed in the surgical package; and
memory disposed in the surgical package storing instructions configured to cause the at least one data processor to perform operations comprising:
catalogue components of the surgical instrument;
determine disposal procedures for each of the components of the surgical instrument; and
provide the disposal procedures at an end of the surgical procedure.

2. The surgical system of claim 1, wherein the operations further comprise:
track the disposal procedures for each component of the surgical instrument;
determine an accuracy level of the disposal procedures for each tracked component; and
provide the accuracy level.

3. The surgical system of claim 1, wherein the operations further comprise:
receive data characterizing available disposal options at a healthcare site hosting the surgical procedure;
adjust the disposal procedures based on the received data; and
provide the adjusted disposal procedures.

4. The surgical system of claim 1, wherein the operations further comprise:
receive data characterizing a geopolitical location of a healthcare site hosting the surgical procedure;
adjust the disposal procedures based on the received data; and
provide the adjusted disposal procedures.

5. The surgical system of claim 1, wherein the operations further comprise:
receive data characterizing a performance error of the surgical instrument;
adjust the disposal procedures based on the received data; and
provide the adjusted disposal procedures.

6. The surgical system of claim 5, wherein the adjusted disposal procedures include a return address for the surgical instrument.

7. The surgical system of claim 1, wherein the disposal procedures include instructions for disassembly, compartmentalization, and/or separation of the catalogued components of the surgical procedure.

8. The surgical system of claim 1, wherein the disposal procedures are provided via a display located on the manufacturer-sealed sterile surgical package.

9. The surgical system of claim 8, wherein the at least one display comprises at least one e-ink label.

10. The surgical system of claim 1, wherein the disposal procedures are provided via a colored LED located on the manufacturer-sealed sterile surgical package, a color of the colored LED corresponding to a color of one or more disposal bins located within an operating room hosting the surgical procedure.

11. The surgical system of claim 1, wherein the disposal procedures are transmitted and displayed on a remote device in electronic communication with the manufacturer-sealed sterile surgical package.

12. A method, comprising:
cataloguing, by a manufacturer-sealed sterile surgical package, components of a surgical instrument contained within the manufacturer-sealed sterile surgical package and used in a surgical procedure;
determining disposal procedures for each of the components of the surgical instrument; and
providing the disposal procedures at an end of the surgical procedure.

13. The method of claim 12, further comprising:
tracking the disposal procedures for each component of the surgical instrument;
determining an accuracy level of the disposal procedures for each tracked component; and
providing the accuracy level.

14. The method of claim 13, further comprising transmitting data characterizing at least one warning message when the accuracy level falls below a predetermined threshold.

15. The method of claim 12, further comprising:
receiving data characterizing available disposal options at a healthcare site hosting the surgical procedure;
adjusting the disposal procedures based on the received data; and
providing the adjusted disposal procedures.

16. The method of claim 12, further comprising:
receiving data characterizing a geopolitical location of a healthcare site hosting the surgical procedure;
adjusting the disposal procedures based on the received data; and
providing the adjusted disposal procedures.

17. The method of claim 12, further comprising:
receiving data characterizing a performance error of the surgical instrument;
adjusting the disposal procedures based on the received data; and
providing the adjusted disposal procedures.

18. The method of claim 17, wherein the adjusted disposal procedures include a return address for the surgical instrument.

19. The method of claim 12, wherein the disposal procedures include instructions for disassembly, compartmentalization, and/or separation of the catalogued components of the surgical procedure.

20. The method of claim 12, wherein the disposal procedures are provided via a display located on the manufacturer-sealed sterile surgical package.

21. The method of claim 12, wherein the disposal procedures are provided via a colored LED located on the manufacturer-sealed sterile surgical package, a color of the colored LED corresponding to a color of one or more disposal bins.

22. A non-transitory computer program product storing instructions which, when executed by at least one data processor forming part of at least one computing system, cause the at least one data processor to implement operations comprising:
cataloguing, by a manufacturer-sealed sterile surgical package, components of a surgical instrument contained within the manufacturer-sealed sterile surgical package and used in a surgical procedure;
determining disposal procedures for each of the components of the surgical instrument; and
providing the disposal procedures at an end of the surgical procedure.
